(19) United States
(12) Patent Application Publication
(10) Pub. No.: US 2009/0280500 A1
(43) Pub. Date: Nov. 12, 2009

(54) Assay for generation of a lipid profile using fluorescence measurement

(75) Inventors: Gareth Royston Jones, Altrincham (GB); David Thomas Clarke, Warrington (GB)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Science And Technology Facilities Council, Warrington (GB)

(21) Appl. No.: 11/792,646

(22) PCT Filed: Dec. 12, 2005
(86) PCT No.: PCT/GB05/04757

§ 371 Date: Jun. 7, 2007

(30) Foreign Application Priority Data
Dec. 11, 2004 (GB) 0427189.6
Dec. 11, 2004 (GB) 0427191.2
Dec. 11, 2004 (GB) 0427192.0

(51) Int. Cl. G01N 33/53; C12Q 1/60; C12M 1/34

(52) U.S. Cl. 435/7.1; 435/11; 435/287.1

(57) ABSTRACT

The present invention relates to a method of generating a lipid profile for a sample solution. The method comprising: a first step of determining the concentration of total lipoprotein in a first aliquot of the sample using fluorescence analysis; a second step of determining the concentration of total cholesterol in a second aliquot of the sample using fluorescence analysis; and optionally a third step of determining the concentration of HDL in a third aliquot of the sample using fluorescence analysis. The concentrations of the total lipoprotein, and of total cholesterol may be used to calculate other lipid components and thereby generate a lipid profile. The invention also concerns apparatus that may be used to perform the method of the invention.

The present invention relates to an assay system for discriminating between different classes of lipid molecules in a sample mixture. In particular, the invention relates to a method of determining the concentration of particular lipids in blood plasma or serum in order to generate a lipid profile. The invention further relates to an apparatus for carrying out the method.

Lipids are a diverse group of organic compounds occurring in living organisms. They are insoluble in water, but soluble in organic solvents. Lipids are broadly classified in to two categories: (i) complex lipids; and (ii) simple lipids. Complex lipids are esters of long-chain fatty acids and include glycerides, glycolipids, phospholipids, cholesterol and waxes. Simple lipids, which do not contain fatty acids, include steroids (for example, cholesterol) and terpenes.

Lipids can combine with proteins to form lipoproteins, which is the form in which lipids, such as cholesterol and triglycerides, are transported in blood and lymph. The lipoproteins found in blood plasma fall into three main classifications: (i) high density lipoproteins (HDL), (ii) low density lipoproteins (LDL), and (iii) very low density lipoproteins (VLDL), together with intermediate density lipoproteins (IDL). For brevity, the term “serum” is used herein, but references to “plasma” should be interpreted as references to plasma or serum.

It is well documented that there is a strong relationship between the concentration of the various lipoproteins in blood plasma and the risk of atherosclerosis, i.e. the development of harmful plaques on blood vessel walls, which can lead to a heart attack. It is also known that the different classes of lipoproteins (HDL, LDL and VLDL) each play a different role in atherosclerosis. For instance, HDL is regarded as being anti-atherogenic whereas LDL is known to be highly atherogenic (the cholesterol it carries correlating closely with atheroscleroses development). VLDL is considered to be slightly atherogenic, and of more significance in females.

Therefore, knowledge of the relative concentrations of each of the various lipid components in the blood (cholesterol, triglycerides, and the lipoproteins, in particular LDL) would be advantageous, as this would assist a clinician in treating patients having blood concentrations of these lipids, which are inappropriate. It will be appreciated that having a knowledge of the patient's lipid profile would be most advantageous to the clinician.

Assays have been developed for determining the concentrations of some of the lipid components in blood. Such assays normally involve initially taking a blood sample from a patient, which is then sent to a clinical laboratory for analysis. Such assays have to be carried out using expensive equipment and take a considerable length of time to generate results. This delays treatment. Furthermore, the tests are involved, and are therefore expensive. In addition, the equipment used in the lab is not readily portable and so cannot be used by GPs, or nurses, carrying out house calls, or even as test kits for home use. Devices have recently been developed that attempt to reproduce lab assays at “point of care” but these have proved to be expensive and require an expert user to operate. Accordingly, there is a requirement to provide improved methods for analysing the lipid profile in blood sera, and simple and relatively inexpensive equipment for carrying out such methods.

Blood serum is a complex mixture of a variety of proteins, and although methods for separating and directly measuring the concentration of the different classes of lipoproteins are known, such methods are complex and expensive. Accordingly, one method of lipoprotein assay widely used in clinical laboratories is an indirect method in which the important LDL concentration is calculated from the measurement of total cholesterol concentration, triglyceride concentration and HDL concentration using the Friedewald equation:—

(CH-LDL)=CH−(CH-HDL)−TG/5

where CH is the total cholesterol concentration, (CH-LDL) is the cholesterol LDL concentration, (CH-HDL) is the cholesterol HDL concentration, and TG is the triglyceride concentration (including background levels of free glycerol).

The HDL, total cholesterol and triglyceride concentrations must be first determined before the LDL concentration can be calculated. It will be appreciated that any errors in the measurement of the HDL, cholesterol or triglyceride concentrations will be compounded in the calculation of the LDL concentration. In addition, the conventional measurement of triglyceride concentration does not discriminate between triglycerides and free glycerol, concentrations of which can vary introducing a further error into the calculation of the LDL concentration. Thus, the calculation of LDL concentration inherently includes errors, which can be extremely significant. Such errors are a particular problem in, for instance, monitoring the progress of widely used LDL decreasing treatments (such as diets, medicine etc) in which it is necessary to accurately monitor relatively small decreases in LDL concentration (typically of the order of several percent) whilst triglyceride levels may be changing dramatically. This is particularly poignant in the treatment of patients with a history of coronary heart disease.

An alternative method for assaying the lipoprotein concentration of blood sera is disclosed in WO 01/53829A1. This document relates to the use of a particular organic luminophore, 4-dimethylamino-4′-difluoromethyl-sulphonyl-benzylidene-acetophenone (DMSBA), as a fluorescent probe. The formula of the probe, identified as K-37, is given below:—

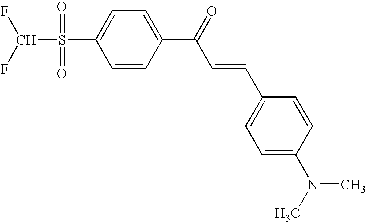

The probe K-37 is not luminous in water, but is highly luminous in aqueous lipoprotein solutions, such as blood serum. In particular, the intensity of the fluorescence is highly dependent upon the lipoprotein content of the blood serum and thus K-37 can be used as a fluorescent probe to measure the concentration of lipoproteins that may be present, i.e. K-37 fluoresces when bound to the lipids of lipoproteins and is excited at appropriate radiation wavelengths. Accordingly, measurement of the time-resolved fluorescence decay of a lipoprotein mixture can be used to give direct information as to the relative concentrations of the different lipoproteins (LDL, and VLDL) present in that mixture.

However, problems with using K-37 time-resolved fluorescence decay is that its measurement is complex and requires expensive equipment. Furthermore, it involves highly technical computer analysis of the data produced, which can be time-consuming to interpret correctly. Accordingly, use of K-37 time-resolved fluorescence decay to determine the concentrations of lipid components in blood has serious limitations for a clinician when wishing to decide a course of treatment.

Therefore, even though there are methods available for determining the concentration of specific lipoproteins in a sample, by using time-resolved fluorescence analysis with the probe K-37, it will be appreciated that this method has a number of limitations.

When a clinician wishes to obtain a comprehensive lipid profile it is important that cholesterol, as well as lipoprotein, is quantified. Cholesterol is mainly transported in the bloodstream in the form of LDLs, and is removed from the blood by means of LDL receptors in the liver. Lack of LDL receptors, occurring as a genetic defect in some individuals, is believed to be a cause of high levels of cholesterol in the blood of affected individuals, predisposing them to atherosclerosis.

The Liebermann-Burchard (L-B) reaction assay is a well-known method for the measurement of total cholesterol in blood, and is considered to be the ‘gold standard’. In a typical L-B assay the reaction reagents are first prepared (e.g. consisting of a solution of 30% glacial acetic acid, 60% acetic anhydride, and 10% sulphuric acid). Secondly, 5 ml of this L-B reagent is then added to 0.2 ml of a sample derived from blood plasma, which are mixed together and then allowed to stand for 20 minutes. The L-B reaction is usually carried out on a sample comprising cholesterol that has been extracted from plasma into an organic solvent. The products of the L-B reaction are two coloured products, which may be measured using conventional absorbance measurements. The absorbance of the products, the concentration of which are related to the concentration of cholesterol, is then measured using a spectrophotometer. The total concentration of cholesterol may be determined from a calibration curve of absorbance against cholesterol concentration, using cholesterol standards (Burke et al., Clin. Chem. 20(7), 794-801 (1974)).

However, problems with the L-B reaction assay are that it requires relatively large quantities of reagents, which is a distinct disadvantage, as the reagents are very corrosive and require special care. It is also normally required that cholesterol is extracted from plasma and this extraction step constitutes a cumbersome extra step in the assay. Accordingly, the L-B reaction assay has been superseded in many laboratories by enzyme-linked assays, because of the requirement for fairly large sample quantities and the use of the corrosive reagents in the L-B reaction assay. However, use of such enzyme-linked assays for determining the concentration of total cholesterol, tend to be easier and safer to carry out, but are less accurate than the L-B assay. Given the results generated are less accurate, clinicians would prefer L-B reaction assay accuracy especially when determining a course of treatment for individuals with higher coronary heart disease risk factors.

Therefore, even though there are methods available for determining the concentration of cholesterol in blood samples, it will be appreciated that these methods also have a number of limitations.

It is therefore an aim of embodiments of the present invention to obviate or mitigate the problems with the prior art, and to provide improved methods for determining a lipid profile for an individual. Such methods involve improved means of measuring the concentration of at least total lipoprotein and cholesterol in a sample taken from the individual. It is a further aim to provide an apparatus for carrying out said method.

According to a first aspect of the present invention, there is provided a method of generating a profile of lipids contained within a sample, the method comprising the steps of:—
(i) determining the concentration of total lipoprotein in a first aliquot of the sample using fluorescence analysis;
(ii) determining the concentration of total cholesterol in a second aliquot of the sample using fluorescence analysis; and
   using the concentrations of the total lipoprotein, and of total cholesterol to generate a lipid profile.

By the term “total lipoprotein”, we mean the collective concentration of at least VLDL, HDL, LDL, IDL and chylomicrons in the sample.

By the term “total cholesterol”, we mean the total concentration of cholesterol in the sample.

By the term “lipid profile”, we mean the concentrations or relative concentrations of lipid components (i.e. total lipoproteins and total cholesterol) in the sample.

Hence, the method according to the first aspect determines the concentration of total cholesterol and total lipoprotein (VLDL, HDL, LDL, IDL, and chylomicrons) in the sample. It is assumed that most lipids present in the sample are bound to lipoproteins. Therefore, it is assumed that the total lipoprotein concentration in the sample is equal to the concentration of total lipids (TL) in the sample.

By the term “total lipids” (TL), we mean the total concentrations of lipid components (i.e. total cholesterol, and triglycerides) in the sample.

It is assumed that the total lipid concentration (TL) is equal to the concentration of triglycerides plus the concentration of the total cholesterol and its esters. Hence, it is possible to calculate the concentration of triglycerides in the sample from the following equation:—

TG=[TL]−[CH]

where CH is the total cholesterol concentration, TL is the total lipid concentration, and TG is the triglyceride concentration, by subtracting the total cholesterol concentration (step (ii)) from the total lipid concentration (which is equal to total lipoprotein concentration—step (i)).

Conventional tests conducted in clinical labs do not measure total lipoprotein. Hence, conventionally, it is required to first determine, and then add the concentration of cholesterol to that of triglyceride to determine the total lipoprotein concentration. However, the inventors of the method according to the invention have found that the conventional measurement of triglyceride in the clinical lab is subject to substantial errors because it relies on the measurement of glycerol, which circulates naturally in the blood. Hence, advantageously, the method according to the invention is not subject to this error, because the number (volume) of lipoprotein particles is measured directly using step (i) to determine the concentration of total lipoprotein, which equates to the total lipid concentration. Hence, errors in the triglyceride concentration caused by circulating glycerol in the sample are obviated.

The sample may be a foodstuff, for which the lipid profile is required. Preferably, the sample is a biological sample, which may be obtained from a subject to be tested. The “subject” may be an mammal and is preferably a human subject. The sample may comprise any biological fluid, for example, blood serum or plasma, or lymph. It is especially preferred that the sample comprises blood serum.

### Step (i)

Step (i) of the method according to the first aspect comprises adding to the first aliquot of the sample a probe substance, which binds to the lipoproteins, and which when bound thereto, fluoresces under appropriate excitation.

It is preferred that the probe substance is K-37. The inventors have developed a simplified assay based on the use of K-37 for measuring lipoproteins in a biological molecule that is particularly useful when a clinician wishes to quickly and efficiently obtain a lipid profile. For determining the concentration of total lipoprotein (i.e. HDL, LDL, and VLDL) in a blood sample using K-37 fluorescence measurements, the inventors realised that it would be preferred that the fluorescence response from the probe substance bound to the various lipoprotein classes must be substantially the same for a given total lipoprotein concentration, i.e. total lipoprotein concentration, irrespective of its composition (i.e. the ratio of HDL:LDL:IDL:VLDL in the sample). Accordingly, it is preferred that K-37 is used in such a manner that the response of fluorescence intensity from the probe substance is substantially linear across the range of concentrations of lipoprotein molecules that would be expected from samples that would be encountered in clinical tests.

While the inventors do not wish to be bound by any hypothesis, they believe that the intensity of fluorescence from the probe substance will depend on its affinity for a particular lipoprotein molecule (HDL, LDL, IDL or VLDL) in the sample, the quantum yield of fluorescence depending on the environment within that lipoprotein molecular complex, and also the degree of fluorescence quenching caused by energy transfer between probe molecules packed closely together. Hence, the inventors reasoned that it may be possible to select a suitable concentration of the probe substance that may be used to make an accurate measurement of total lipoprotein by simple fluorescent measurement. The inventors further realised that such a concentration of probe would preferably balance K-37's higher quantum yield in HDL compared to VLDL and LDL with its higher affinity for HDL, and therefore a higher degree of quenching within HDL to produce a constant fluorescence signal response over all lipoprotein particles.

The inventors therefore conducted a series of experiments to investigate whether it was possible to obtain a linear and equal relationship between the fluorescence of the probe substance, K-37, and the lipoprotein concentration for each lipoprotein particle type (HDL, LDL, and VLDL), across the range of lipoprotein concentrations that would be encountered in real serum samples. To their surprise, they found that there was a defined concentration of K-37 at which there was a linear relationship between the fluorescence of K-37 and lipoprotein concentration.

Hence, it is preferred that the concentration of total lipoprotein in sample solution is measured by adding to the first aliquot of the sample between 0.1 mM-1.0 mM of K-37, which binds to lipoproteins in the sample, and which when so bound fluoresces under appropriate excitation; and determining the total lipoprotein concentration in the sample using fluorescence analysis.

Advantageously, at the concentration of 0.1-1.0 mM K-37, a more accurate determination of the concentration of the total lipoprotein is possible, as there is surprisingly considerably less signal distortion obtained from analysis of the fluorescence measurements.

Suitably, the concentration of K-37 added to the sample may be between approximately 0.2-1.0 mM, more suitably, between approximately 0.3-0.9 mM, and even more suitably, between approximately 0.5-0.8 mM. Preferably, the concentration of K-37 added to the sample is between approximately 0.65-0.75 mM. 0.65 mM K-37 is a preferred concentration and an especially preferred concentration is about 0.7 mM K-37. The inventors have found that 0.65 mM K-37 is useful in a number of experimental conditions although 0.7 mM represents the preferred concentration of this probe when biological samples, which contain proteins, are assayed.

Hence, in a preferred embodiment, approximately 0.7 mM of the probe substance, K-37, is added to the first aliquot in step (i) of the method according to the invention.

Preferably, step (i) involves exciting the sample at an excitation wavelength of between about 400 nm-500 nm, and more preferably, between about 420 nm-480 nm, and even more preferably, between about 440 nm-470 nm. An especially preferred excitation wavelength of about 450 nm may be used although excitation at any wavelength between about 450-470 nm is also particularly preferred.

Preferably, the method comprises observing the fluorescence at an emission wavelength of between about 500-650 nm, and more preferably, between about 520 nm-620 nm. An especially preferred emission wavelength of about 540 nm (or higher) may be used, at which the most accurate readings for determining the total lipoprotein concentration (i.e. the concentration of HDL, IDL LDL and VLDL, but also chylomicrons if present) may be observed.

### Step (ii)

The conventional “gold standard” method used for determining the concentration of cholesterol in a sample is by using the Liebermann-Burchard (L-B) reaction assay, which is an absorbance-based assay. A schematic showing the L-B reaction is provided as FIG. 8.

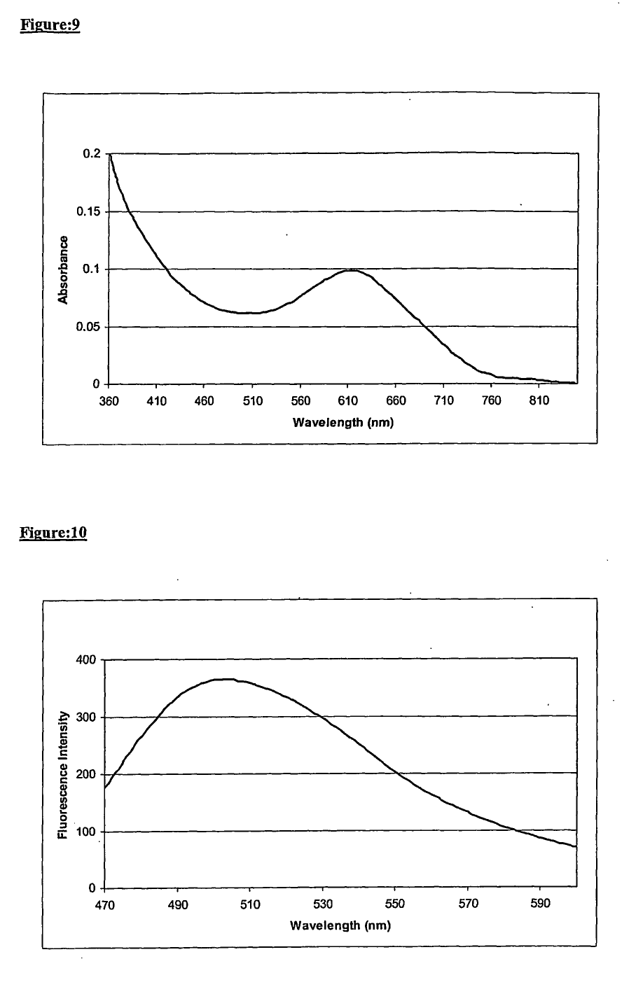

The inventors investigated the L-B reaction and were surprised to find that the product of the L-B reaction fluoresced. The traditional L-B assay was undertaken by measuring the absorbance of the band at or around 600 nm (see FIG. 9). However, when exciting the absorption bands at shorter wavelengths (i.e. less than about 500 nm) the product of the L-B reaction was surprisingly highly fluorescent. FIG. 10 shows the fluorescence emission spectrum of the L-B product, and it was surprisingly found that the fluorescence extends over the range of 470-600 nm.

Accordingly, step (ii) of the method according to the invention comprises adding to the second aliquot of the sample reagents, which cause the Liebermann-Burchard (L-B) reaction. However, instead of measuring the absorbance of the coloured product at 600 nm and longer wavelengths, as in the conventional L-B reaction assay, step (ii) of the method according to the invention comprises measuring fluorescence to determine the cholesterol concentration in the sample.

It is preferred that step (ii) comprises determining the concentration of total cholesterol by: adding to the sample reagents which cause the Liebermann-Burchard (L-B) reaction; and determining the total cholesterol concentration in the sample using fluorescence analysis.

Another benefit of this step (ii) is that liquid samples (e.g. serum or plasma) may be used directly in the method. This is in contrast to a conventional L-B reaction assay that may require extraction of cholesterol from the primary sample before it is combined with the L-B reagents.

Preferably, the reagents added to the sample result in the reaction of all of the total cholesterol present in the sample, i.e. the cholesterol and esters thereof, which may be associated with lipoproteins (e.g. LDL or HDL), which may be present in the sample. The L-B reagents preferably add double bonds to cholesterol in the sample, as is illustrated in FIG. 8. Accordingly, by the term “reagents which cause the L-B reaction”, we mean reagents which, when added to a sample containing cholesterol, cause or induce the cholesterol in the sample to be increasingly unsaturated.

Step (ii) may employ the same reagents as the generic Liebermann-Burchard reaction assay (L-B), or other assays for determining the concentration of cholesterol, which may be based on the L-B reaction, for example, the Abell-Kendal assay, which will be known to the skilled technician (Abell et al., J. Biol. Chem. 195 (1) p 357-366). However, instead of measuring the absorbance of the coloured products at 550 nm as in the conventional L-B reaction assay, step (ii) comprises measuring fluorescence to determine the cholesterol concentration in the sample.

The L-B reaction reagents may comprise three different reagents,

The first reagent comprises a cholesterol solvent. Examples of suitable cholesterol solvents include acetic acid, dioxane, and/or chloroform. Preferably, the cholesterol solvent comprises glacial acetic acid.

The second L-B reaction reagent is a strong acid. The inventors do not wish to be bound by any hypothesis but believe the acid performs an elimination reaction that extracts water from cholesterol leaving a higher degree of conjugation. The inventors believe it is this conjugation (i.e. an increased number of double bonds) that fluoresces when excited according to the invention. The strong acid is preferably an oxo acid (X—OH) such as phosphoric acid (H₃PO₄) and more preferably H₂SO₄. The acid may also be HNO₃, H₂SeO₄, HClO₄, and HMnO₄. Alternatively the acid may be a lewis acid such as Al₂Cl₆, SnCl₄ and FeCl₃ or titanium dioxide.

It is most preferred that the strong acid comprises sulphuric acid, preferably, at a concentration of about 3-20% (v/v), or Al₂Cl₆ at a concentration of about 0.5-2.5 Molar.

The third L-B reaction reagent comprises acetic anhydride. It is preferred that the acetic anhydride to solvent ratio is between 0.25:1 to 10:1, more preferably, between 0.5:1 to 5:1, and even more preferably, between 1:1 to 3:1. In a preferred embodiment, the acetic anhydride to solvent ratio is about 2:1.

It is preferred that the L-B reagents comprise about 30% (v/v) glacial acetic acid, about 60% (v/v) acetic anhydride, and about 10% (v/v) sulphuric acid.

In addition, the L-B reagents may also comprise additives such as, anhydrous sodium sulphate, or sodium salicylate etc., which are used to stabilise the reagent for longer-term storage. The additives may be added in the range of about 0.5-3% (v/v).

Step (ii) preferably comprises the step of exciting the second aliquot of the sample (i.e. the product of the L-B reaction) at an excitation wavelength below about 500 nm, and more preferably, below about 470 nm. An especially preferred excitation wavelength of 450 nm or shorter wavelengths may be used in order to cause the product of the L-B reaction to fluoresce.

The resultant fluorescence may then be observed and measured at an emission wavelength of between 500-650 nm, and more preferably, between 520-600 nm. An especially preferred emission wavelength of 540 nm may be used.

Accordingly a preferred step (ii) comprises adding L-B reagents (e.g. about 30% (v/v) glacial acetic acid, about 60% (v/v) acetic anhydride, and about 10% (v/v) sulphuric acid) to a second aliquot of the sample; exciting the sample at about 450 nm and measuring fluorescence at an emission wavelength of about 540 nm or above.

An advantage of measuring the fluorescence of the product of the L-B reaction, instead of measuring its absorbance as in conventional methods, is that much smaller volumes of the reagents are required. This is particularly advantageous as the reagents of the L-B assay are very corrosive and therefore dangerous to use. Hence, reducing the amount of reagents required in the fluorescence assay of step (ii) of the method is much safer for a technician than the conventional L-B absorbance assay. Using smaller volumes of reagents also means that a smaller device can be used for carrying out the assay. In addition, measuring fluorescence is much more sensitive than measuring absorbance. Therefore, it is possible to make a more precise determination of the cholesterol concentration using fluorescence than by measuring absorbance.

It will be appreciated that the excitation wavelength used for determining the concentration of total lipoprotein in step (i) of the method according to the invention, and the concentration of total cholesterol in step (ii) of the method, is 450 nm in both cases. In addition, the emission wavelength used for both measurements is 540 nm. Hence, both parameters (total cholesterol and total lipoprotein) may be determined simultaneously and quickly. This is a considerable improvement over conventional assays, which have to be carried out separately causing a delay in the generation of results. In addition, the fact that both parameters can be measured at the same time also simplifies the instrumentation required to carry out the measurements. This allows for the design of apparatus, as discussed below, that may generate lipid profiles in surgeries or even at home and obviates the need to assay samples in dedicated laboratories.

Therefore, it will be appreciated that the method according to the invention may be used to quickly and accurately determine the concentrations of total lipoprotein in the sample using step (i), and also determine the concentration of total cholesterol and its esters in the sample using step (ii). In addition, the concentration of triglyceride may be calculated by subtracting total cholesterol concentration from the total lipoprotein concentration as discussed above. Hence, a more detailed lipid profile of the sample is thereby generated consisting of total lipoprotein concentration, total cholesterol concentration, and also triglyceride concentration, which would be useful to the clinician.

Further embodiments of the method of the first aspect of the invention may include the following:
Step (iii): Discrimination Between Types of Lipoprotein in a Sample

The inventors realised that a more detailed lipid profile may be obtained if they could employ a further assay step that discriminates between lipoproteins in the sample being tested Therefore, the inventors investigated the use of probe substances other than K-37 to see if it was possible to distinguish between the various lipoprotein molecules. They were surprised to find that a number of dyes will bind to lipoproteins and will exhibit different fluorescent responses that are dependant on the particular lipoprotein bound. Fluorescent measurements with these dyes makes it possible to distinguish between the types of lipoprotein present in a sample. This is done by comparing the enhanced or reduced fluorescence caused by one type of lipoprotein in a lipoprotein mixture with the fluorescence expected from the other lipoproteins (in the absence of the specific propertied lipoprotein) as determined from a calibration curve and a known value of the total lipoprotein content given by the K-37 assay of step (i).

For example the fluorescent dye, Nile Red, exhibited a significantly higher fluorescence in HDL than in the other lipoproteins, such as LDL and VLDL. Therefore, the inventors realised that a second probe substance (e.g. Nile Red, or any other lipophilic probe that shows specificity, or fluorescence enhancement or reduction towards a particular lipoprotein), may be used to discriminate between classes or subclasses of lipoproteins in the sample. This is possible after the total lipid concentration has been determined according to step (i).

Accordingly the method of the invention may further comprise the step of:
(iii) determining the concentration of a particular class, or sub-class of lipoprotein in a third aliquot of the sample using fluorescence analysis.

Step (iii) preferably involves determining the concentration of a particular class, or sub-class of lipoprotein by the shift in fluorescence response of a dye specific to that lipoprotein using a second probe.

The second probe substance may be added to the third aliquot of the sample, which probe binds to a specific class or subclass of lipoproteins and which when bound thereto, modifies the fluorescence yield under appropriate excitation, which is indicative of the concentration of the specific class or sub-class of lipoproteins.

It is preferred that step (iii) comprises adding the probe Nile Red to the third aliquot of the sample and then utilising the results from step (i) of the method to determine the concentration of HDL in the sample.

Preferably, in order to determine the HDL concentration in the sample using Nile Red, a calculation must be made of the excess fluorescence from Nile Red due to the presence of HDL. Firstly, the total lipoprotein concentration (measurement “A”) is measured by the linear correlation of K-37 fluorescence with lipoprotein concentration (as determined by step (i)).

Secondly, Nile Red fluorescence is then calibrated with LDL (and/or VLDL as the fluorescence to concentration response must be essentially the same) at various concentrations to obtain a calibration curve with slope “X” and intercept “Y”. A skilled technician would know how to prepare a range of concentrations of LDL (and/or VLDL), and determine the respective fluorescence for each concentration.

Thirdly, an additional calibration curve is then constructed for a series of concentrations of HDL and a constant concentration of LDL to give slope “Z”. Fourthly, knowing the total lipoprotein concentration from the K-37 measurement “A” and the excess Nile Red fluorescence of the unknown sample “B”, the concentration of HDL “C” in the unknown sample can be determined by the following equation:—

C=(B−(AX−Y))/Z

It will be appreciated that in the practice of the invention that pre-prepared or standard calibration curves may be used. Furthermore devices developed to generate lipid profiles (see below) may have internal standards and/or have processing means that will allow for automatic calculation of HDL levels without user intervention.

Therefore, the method according to the invention may further comprise determining the concentration of HDL in the sample using fluorescence analysis. The method comprises a further step (step (iii)) in which the probe substance Nile Red is added to a third aliquot of the sample, which probe binds to HDL and other lipoproteins. Under appropriate excitation Nile Red fluoresces more and more strongly in proportion to the concentration of HDL in the sample. When this additional step is carried out in the method of the invention, an even more detailed lipoprotein profile of the sample may be generated consisting of total lipoprotein concentration, and HDL concentration, which would be very useful in clinical assessment of the subject.

The inventors conducted a series of experiments to determine the optimum concentration of Nile Red, which should be added to the sample, to improve the accuracy of the determination of HDL in the sample, and this required considerable inventive endeavour. Accordingly, the concentration of the probe substance Nile Red added to the sample may be between approximately 0.1-1 mM. Advantageously, at this concentration of Nile Red, a more accurate determination of the concentration of the HDL concentration is possible.

Suitably, the concentration of Nile Red added to the third aliquot of the sample may be between approximately 0.1-0.9 mM, more suitably, between approximately 0.2-0.7 mM, and even more suitably, between approximately 0.3-0.6 mM. It is especially preferred to add Nile Red to the sample to a final concentration of about 0.4 mM.

The fluorescence of Nile Red is preferably induced by exciting the sample at an excitation wavelength of between about 400 nm-650 nm.

It is preferred that the excitation wavelength is 400 nm-650 nm; preferably, between about 420 nm-620 nm, more preferably, between about 500 nm-610 nm and even more preferably, between about 590 nm-610 nm. An excitation wavelength of about 600 nm may be used in connection with Nile Red which gives the largest discrimination (5×) between the fluorescence response from Nile Red in HDL when compared with the other lipoproteins.

The resultant fluorescence from Nile Red may then be observed and measured at an emission wavelength of between about 540-700 nm, and more preferably, between about 570-650 nm. A preferred emission wavelength of about 620 nm may be used, at which the most accurate readings for determining the concentration of HDL may be observed.

Therefore, it will be appreciated that fluorescent measurements may be used for determining the concentration of HDL, total lipoprotein, and also total cholesterol. All three parameters (total cholesterol, total lipoprotein, and HDL) may be determined simultaneously and quickly by exciting and measuring fluorescence over a very similar range of wavelengths. As discussed above, this is a considerable improvement over conventional assays, which have to be carried out separately, and often in a dedicated laboratory, causing a delay in the generation of results. In addition, the fact that all three parameters can be measured at the same time considerably simplifies the instrumentation required to carry out the measurements.

### Influence of Human Serum Albumin on the Determination of Lipoprotein

The inventors investigated whether it was possible to further improve the accuracy of the individual assays used in steps (ii) and (iii) of the method according to the invention, and so turned their attention to Human Serum Albumin (HSA), which is a major component of blood serum, having a concentration of approximately 30-50 mg/ml.

HSA is known to have at least two types of binding site that are capable of binding various ligands. A first type is referred to herein as “a hydrophobic domain” whereas a second type of domain is referred to herein as a “drug binding domain”. These domains are known to one skilled in the art and are distinguished from each other in a paper in Nature Structural Biology (V 5 p 827 (1998)). This paper identifies a hydrophobic domain as one to which fatty acids may bind whereas the drug binding domain is capable of binding a number of drugs that may be associated with HSA.

From their experiments, the inventors have surprisingly established that the probe substances K-37 and Nile Red may both individually bind to hydrophobic binding sites/domains of HSA. Hence, K-37 and Nile Red are both ligands for HSA. In addition, surprisingly, the inventors found that K-37 and Nile Red fluoresce when bound to HSA. Therefore, while the inventors do not wish to be bound by any hypothesis, the inventors believe that this additional fluorescence of K-37 when bound to HSA may cause a substantial background signal, which may distort and lead to significant errors in the determination of concentration of total lipoprotein in step (i) of the method according to the invention. Similarly, the inventors believe that this additional fluorescence of Nile Red when bound to HSA may cause a substantial background signal, which may distort and lead to significant errors in the determination of concentration of HDL when step (iii) is used in the method according to the invention.

As a result, the inventors investigated the effects of inhibiting the binding of the ligand K-37, and the ligand Nile Red, with HSA. In particular, they attempted to block the hydrophobic binding sites of HSA at which the probes K-37 and Nile Red bind and fluoresce. This work is described in Examples 3 and 4. While the inventors do not wish to be bound by any hypothesis, to their surprise, they found that inhibiting the binding of the ligand K-37 with the hydrophobic binding sites resulted in the fluorescence of the probe substance when bound to the lipoprotein molecules (HDL, LDL, VLDL) being a more accurate measure of the concentration of total lipoprotein in the sample than if no ligand binding inhibitor was added. The inventors also found that inhibiting binding of the ligand Nile Red to HSA improved the accuracy of the HDL determination.

Accordingly, it is preferred that the method according to the invention comprises adding to the first aliquot, and third aliquot if appropriate, of the sample a ligand binding inhibitor that is adapted to substantially inhibit the binding of the probe substances (K-37 and/or Nile Red) to HSA and preferably, the hydrophobic binding sites thereof. It is especially preferred that the ligand binding inhibitor is also added to the sample prior to or at the same time as the probe is added to the sample.

The ligand binding inhibitor may be hydrophobic. The inhibitor may be amphipathic. The ligand binding inhibitor may comprise a fatty acid or a functional derivative thereof, as well as other hydrophobic molecules. Examples of suitable derivatives of fatty acid, which may block the hydrophobic binding sites of HSA may comprise a fatty acid, its esters, acyl halide, carboxylic anhydride, or amide etc. A preferred fatty acid derivative is a fatty acid ester.

The fatty acid or derivative thereof may comprise a C₁-C₂₀ fatty acid or derivative thereof. It is preferred that the fatty acid or derivative thereof may comprise a C₃-C₁₈ fatty acid or derivative thereof, more preferably, a C₅-C₁₄ fatty acid or derivative thereof, and even more preferably, a C₇-C₉ fatty acid or derivative thereof.

It is especially preferred that the ligand binding inhibitor comprises octanoic acid (C₈) or a derivative thereof, for example, octanoate. Preferably, the ligand binding inhibitor is added as an alkali metal octanoate, preferably a Group I alkali metal octanoate, for example, sodium or potassium octanoate.

Preferably, between about 10-400 mM of the ligand binding inhibitor is added to the sample prior to carrying out step (i) of the method, more preferably, between about 20-200 mM, and even more preferably, between about 50-150 mM is added. It is especially preferred that about 100 mM of the inhibitor is added. Hence, in a preferred embodiment of the method, about 100 mM of sodium octanoate may be added to the sample before or at the same time as carrying out step (i).

When the method of the invention also extends to the use of Nile Red in a step (iii), it is preferred that between about 10-400 mM of the ligand binding inhibitor is added to the sample, more preferably, between about 20-200 mM, and even more preferably, between about 50-150 mM is added. It is most preferred that about 100 mM of the inhibitor is used. Hence, in a preferred embodiment of the method, it is preferred that about 100 mM of sodium octanoate is added to the third aliquot of sample before adding Nile Red and carrying out the HDL assay according to the method.

In a preferred embodiment of the invention, a ligand binding inhibitor, for example, about 100 mM sodium octanoate, is first added to the first aliquot taken from the sample, with approximately 0.7 mM of the K-37 probe, prior to carrying out the fluorescence measurement of the total lipoprotein concentration in step (i) of the method. In embodiments involving HDL determination, a ligand binding inhibitor, for example, about 100 mM sodium octanoate, is first added to a third aliquot, with approximately 0.4 mM of the Nile Red probe, prior to carrying out the fluorescence measurement of the HDL concentration in the method.

Advantageously, the ligand binding inhibitor combined with the defined concentration of the K-37 probe result in highly accurate measurements of total lipoprotein being obtained (and HDL concentration when Nile Red probe is added), which in turn improves the accuracy of the determination of triglyceride during subsequent calculations (see below).

The inventors have additionally found that Nile Red also interacts with the drug binding domain on HSA that is referred to above. Ligands for this drug binding domain include drug molecules such as: thyroxine, ibuprofen, diazepam, steroid hormones and their derivatives (drugs), haem, bilirubin, lipophilic prodrugs, warfarin, coumarin based drugs, anesthetics, diazepam, ibuprofen and antidepressants (e.g. thioxanthine).

The inventors have found that agents may be used to block this drug binding domain and that this results in further improvement of assay results with Nile Red. The abovementioned drugs, or any other molecule with affinity to this domain, may be used as agents for blocking the drug binding domain of HSA. However it is most preferred that benzoic acid or a derivative thereof (e.g. trichlorobenzoic acid or triiodobenzoic acid) is used to block the drug binding domain. Accordingly a most preferred step (iii) also includes the addition of an agent for blocking the drug binding domain of HSA.

Examples 1 and 4 illustrate how fluorescence measurements of the dye K37 may be used to determine the concentration of total lipoproteins in the sample, and how the fluorescence measurements of Nile Red may be used to determine the concentration of HDL in the sample. Both dyes may be excited at a wavelength of 450 nm and the emission of the dyes may be measured at wavelengths at or longer than 540 nm. Examples 3 and 4 also describe blocking HSA with a ligand (K37 or Nile Red) binding inhibitor in order to improve the accuracy of the results produced by K-37 and Nile Red fluorescence.

In addition, Example 2 describes how fluorescent measurements (i.e. not absorbance) of the product of the L-B assay may be used to determine the concentration of total cholesterol in the sample. Fortuitously, as with K-37 and Nile Red, the product of the L-B reaction may also be excited at a wavelength of 450 nm. In addition, as with K-37, its emission may also be measured at 540 nm. Therefore, the inventors realised that it is possible to create a single method for analysing the lipid composition of a patient's blood sample in order to create a lipid profile for that patient.

Hence, a preferred method consists of two or three fluorescence assays (steps (i), (ii) and (iii)), all of which can be carried out under very similar conditions, and hence, can produce results very quickly. The clinician may use this information to decide upon a certain course of treatment. A most preferred embodiment of the method of the first aspect of the invention may comprise taking a blood sample from a patient, and then separating the blood serum from the blood cells. This may be achieved by known techniques, such as filtration or centrifugation. The plasma may then be separated in to three aliquots, each of which is subjected to fluorescence analysis to determine the concentration of a lipid component. A first aliquot may be used to determine the concentration of total lipoprotein in the sample; a second aliquot may be used to determine the concentration of total cholesterol in the sample; and a third aliquot may be used to determine the concentration of HDL in the sample (knowing the total lipoprotein in the sample from the first aliquot).

To the first aliquot, an HSA ligand binding inhibitor, for example, sodium octanoate, may be added to a final concentration of about 100 mM. It is preferred that the probe K37 is also added to the first aliquot in step (i), preferably to a final concentration of about 0.7 mM. The first aliquot may then be excited at approximately 450 nm in order to cause the probe to fluoresce. The fluorescence may then be measured at an emission wavelength of 540 nm or above. From this value, it is then possible to determine the concentration of total lipoprotein (HDL, LDL, IDL and VLDL and chylomicrons if present) in the sample.

To the second aliquot, adding L-B reagents (e.g. about 30% (v/v) glacial acetic acid, about 60% (v/v) acetic anhydride, and about 10% (v/v) sulphuric acid); exciting the sample at about 450 nm; and measuring fluorescence at an emission wavelength of about 540 nm or above. From this value, it is then possible to determine the concentration of cholesterol in the sample.

To the third aliquot, an HSA ligand binding inhibitor for example, sodium octanoate, may be added to a final concentration of about 100 mM. An agent that will bind to the drug binding domain of HSA such as benzoic acid may also be added, to a concentration between 1-10 mM or more specifically approximately 5 mM. The probe Nile Red may then be added to a final concentration of about 0.4 mM. This sample may then be excited at approximately 600 nm in order to cause the probe to fluoresce. The fluorescence may be measured at an emission wavelength of approximately 620 nm, and from this value it is then possible to determine the concentration of HDL in the sample as described in Example 4.

The triglyceride concentration may be easily calculated by subtraction of the results of steps (ii) from step (i) of the method of the first aspect. Therefore, the lipid profile generated now also includes the concentration of triglycerides, which assists the clinician treating the patient. Hence, the values of four parameters can be determined using the method of the invention.

It is assumed that the concentration of HDL as calculated by assaying the third aliquot is a direct measure of the concentration of cholesterol bound to HDL. Hence, with a knowledge of the total cholesterol concentration determined by assaying the second aliquot, the HDL concentration determined by assaying the third aliquot, and the triglyceride concentration calculated as described in the preceding paragraph, it is then possible to calculate the concentration of cholesterol bound to LDL in the sample by inserting the known values in to the Friedewald Equation:—

(CH-LDL)=CH−(CH-HDL)−TG/5

where CH is the total cholesterol concentration, (CH-LDL) is the cholesterol LDL concentration, (CH-HDL) is the cholesterol HDL concentration, and TG is the triglyceride concentration.

As a result, the lipid profile generated by the method of the invention now also includes the concentration of cholesterol bound to LDL in the sample. It is especially advantageous to know the LDL cholesterol concentration as it is highly atherogenic. Hence, the method provides a multi-readout of at least five parameters of the lipid composition in the sample. Furthermore, it is possible to calculate/estimate the concentration of CH-VLDL from the triglyceride concentrations, as it is generally assumed that most of the triglycerides are carried in VLDL and the cholesterol component of VLDL is 20%. This is particularly advantageous for helping the clinician to decide on a suitable course of treatment.

In addition to developing the method according to the first aspect, the inventors also developed an apparatus for carrying out the method.

Hence, according to a second aspect of the present invention, there is provided apparatus for generating a lipid profile for a sample solution, the apparatus comprising a reaction reservoir for conducting a cholesterol and lipoprotein assays; containment means adapted to contain reagents required for the method according to the first aspect; excitation means operable to excite the sample so that it fluoresces, and detection means operable to detect the fluorescence emitted by the sample.

Preferably the apparatus comprises means for mixing the sample and reagents in the reaction reservoir.

Preferably, the apparatus comprises a number of types of reservoir.

A first type of reservoir may be for containing the sample and is referred to herein as a sample reservoir. There may be a single reservoir from which first, second and optionally third aliquots of the sample may be taken for performing assays according to steps (i), (ii) and optionally (iii) of the method according to the invention. Alternatively there may be a separate sample reservoir for each aliquot of sample. It will be appreciated that, in some embodiments, the device may be designed such that the sample may be directly introduced into the reaction reservoir. This would obviate the need for a sample reservoir.

The reaction reservoir may be a second type of reservoir in which the assay to determine the concentrations of cholesterol and lipoprotein in the sample aliquots may be conducted (following introduction of the sample and reagents). The apparatus may comprise a single reaction reservoir and may be washed out between reactions on different sample aliquots. Alternatively multiple (e.g. single use) reaction reservoirs may be brought into contact with the excitation means. Accordingly there may be a reaction reservoir for each step of the method according to the first aspect of the invention.

The containment means may comprise a third type of reservoir, namely reagent reservoirs. A first reagent reservoir may contain the K-37 dye and other reagents for the total lipoprotein assay (e.g. a ligand binding inhibitor). A second reagent reservoir may contain reagents for the cholesterol assay (i.e. L-B reaction reagents. When HDL is to be determined (e.g. using Nile Red) the containment means may comprise a third reagent reservoir, containing Nile Red dye and other reagents for determining HDL (e.g. a ligand binding inhibitor and an agent for blocking the drug binding domain of HSA). Alternatively the reagents for each assay may be included in separate containment means. Accordingly reagents for the K-37 assay may be within a first reagent reservoir in a first containment means; reagents for the cholesterol assay may be within a second reagent reservoir in a second containment means; and the reagents for the Nile Red assay may be within a third reagent reservoir in a third containment means.

It is preferred that the reaction reservoir is arranged so that it may be brought into optical contact with the excitation means. The reaction reservoir should be arranged such that fluorescence produced from the assay may be detected by the detection means.

In a preferred embodiment the apparatus comprises a single containment means that has three reagent reservoirs. The first reagent reservoir contains the probe substance, K-37, in a suitable diluent and may further contain an HSA ligand binding inhibitor, for example, sodium octanoate. A second reagent reservoir contains L-B reaction reagents. The third reagent reservoir may comprise the probe substance Nile Red and an HSA ligand binding inhibitor, for example, sodium octanoate and, also an agent for blocking a drug binding domain on HSA such as benzoic acid. In use the reagents may be urged into respective reaction reservoirs (also within the single containment means) and mixed with three respective aliquots of the sample. Reaction steps (i), (ii) and (iii) of the method of the first aspect of the invention can then be performed and fluorescence measurements taken.

The apparatus may comprise a reader and preferably, a cartridge adapted to be placed in functional communication therewith. Preferably, the cartridge may be inserted into, or attached to, the reader. The reader may comprise docking means in which the cartridge is inserted. The docking means may be a slot. Hence, preferably, the cartridge is removable from the reader.

The cartridge may comprise the, or each, containment means and the reaction and sample reservoir (if present). Hence, the cartridge carrying the assay reagents may be removed from the reader once the reagents have been exhausted, and replaced with a new cartridge containing new assay reagents. It will be appreciated that a self-contained cartridge (comprising all reservoirs) may be readily used as a single-use reaction cartridge. A cartridge may be simply removed from the reader and replaced with a new cartridge comprising reagents and sample aliquots may be deployed into a reaction reservoir or reservoirs within the cartridge.

The reader may comprise the excitation means and preferably, the detection means.

Preferably, the apparatus comprises processing means adapted to determine the concentration of cholesterol and total lipoprotein in the sample based on the fluorescence detected. In a preferred embodiment, the processing means is also adapted to determine the concentration of HDL in the sample based on fluorescence analysis. The processing means may be adapted to calculate the concentration of LDL, VLDL and IDL, in the sample based on the concentrations of total lipoprotein, and HDL.

The apparatus may comprise display means for displaying the concentrations of cholesterol and total lipoprotein and preferably, the concentration of HDL in the sample, preferably as a read-out. For example, the display means may comprise an LCD screen, or may rely on a computer for powering and/or computing and/or display.

Preferably, the apparatus is portable, and may be used to generate a patient's lipid profile by a taking a sample from a patient and then conducting the assay at the site where the sample is taken.

The apparatus should be adapted to contain a sample that may be any biological fluid, for example, blood, serum, lymph etc.

Preferably, the excitation means comprises an illumination source operable to illuminate the sample at about 400 nm-500 nm (for the cholesterol and K-37 assay) and for preferred embodiments of the Nile Red assay, at about 600 nm. Accordingly the light source is preferably capable of illuminating the sample at between about 400 nm-600 nm. The illumination source may comprise a bulb or one or more LEDs and the excitation wavelengths may be varied utilising a 450 nm interference filter and an interfence filter at 600 nm. The excitation means may comprise polarising means operable to polarise light produced by the illumination source. The excitation means may comprise focussing means adapted to focus the light on to the sample. The focussing means may comprise a lens.

Preferably, the detection means comprises a photodiode or photomultiplier, which is preferably yellow-red sensitive. Fluorescence emitted by the sample is preferably detected at about 500 nm-650 nm, and more preferably, 540 nm, and longer wavelengths, for the cholesterol assay and K-37 assay. The detection means should be able to detect fluorescence emitted at 620 nm for assays involving Nile Red. The fluorescence may be collected by a second lens, and may pass through a polariser. Scattered excitation light may be removed by a cut-off filter. For measurement of the fluorescence intensity, the current from the photodiode or the count rate from the photomultiplier may be read from an ammeter, voltmeter, or rate-meter module.

In one embodiment, the apparatus may comprise a reader that is adapted to receive two or three cartridges (i.e. a cartridge for each assay step—steps (i), (ii) an optionally (iii) of the method of the invention). Such a reader may comprise two (or more) excitation means that can be aligned with each reaction reservoir. In addition, the apparatus may comprise a detection means for each reaction reservoir.

The apparatus may also comprise an excitation correction system so that fluctuations of the light source may be accounted for. The apparatus may comprise at least one fluorescence standard for use in calibrating prior to determine the concentration of lipoprotein. The standard may be an internal standard.

Accordingly, the apparatus is configured to detect and measure the fluorescence intensities of each assay simultaneously or in turn as the cartridge enters the reader or at some time thereafter, to thereby generate the lipid profile consisting of cholesterol, total lipoprotein concentration, and HDL concentration.

Advantageously, the apparatus according to the second aspect may be used to carry out quick and easy assays, which can be conducted simultaneously to generate the lipid profile from the biological fluid. A clinician with knowledge of cholesterol, lipoprotein and HDL concentrations can then decide on an effective course of treatment. In addition, the apparatus is portable and may be used by GPs, or nurses who carry out home visits, or even as test kits for home use.

The apparatus may comprise a fluorescent standard to automatically calibrate the instrument.

The apparatus is configured to detect and measure the fluorescence intensities of each of the three assays simultaneously or in turn as the cartridge enters the reader or at some time thereafter, to thereby generate the lipid profile consisting of total lipoprotein concentration, HDL concentration, and cholesterol concentration. The clinician or an assistant may then calculate the concentration of triglycerides, and also LDL concentration, as described in Example 5.

Advantageously, the apparatus according to the second aspect may be used to carry out quick and easy assays, which can be conducted simultaneously to generate the lipid profile from the biological fluid. The clinician with knowledge of LDL, triglyceride and HDL concentrations can then decide on an effective course of treatment. In addition, the apparatus is portable and may be used by GPs, or nurses who carry out homes visits, or even as test kits for home use.

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying diagrammatic drawings, in which:—

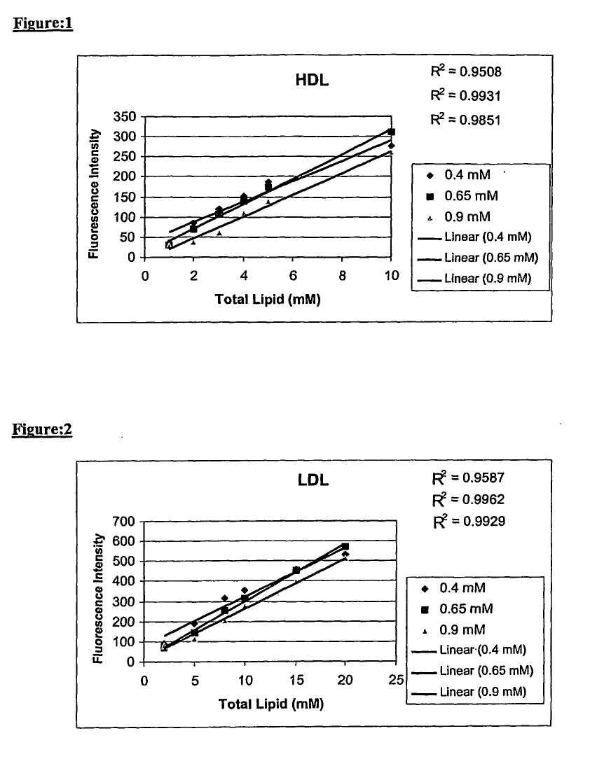

FIG. 1 is a graph showing fluorescence intensity against total lipid concentration for K-37 at three concentrations (0.4 mM, 0.65 mM and 0.9 mM) in HDL as referred to in Example 1;

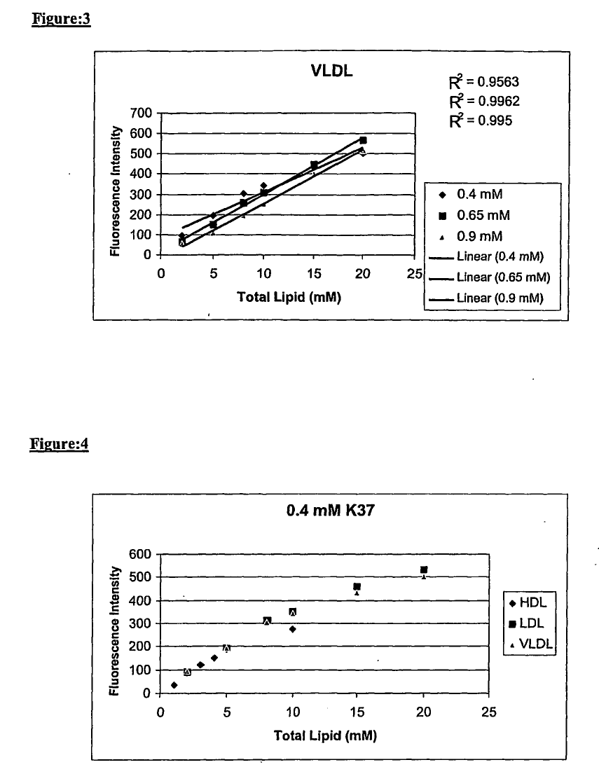

FIG. 2 is a graph showing fluorescence intensity against total lipid concentration for K-37 at three concentrations (0.4 mM, 0.65 mM and 0.9 mM) in LDL as referred to in Example 1;

FIG. 3 is a graph showing fluorescence intensity against total lipid concentration for K-37 at three concentrations (0.4 mM, 0.65 mM and 0.9 mM) in VLDL as referred to in Example 1;

FIG. 4 is a graph showing fluorescence intensity against total lipid concentration for 0.4 mM K-37 in HDL, LDL, and VLDL as referred to in Example 1;

FIG. 5 is a graph showing fluorescence intensity against total lipid concentration for 0.65 mM K-37 in HDL, LDL, and VLDL as referred to in Example 1;

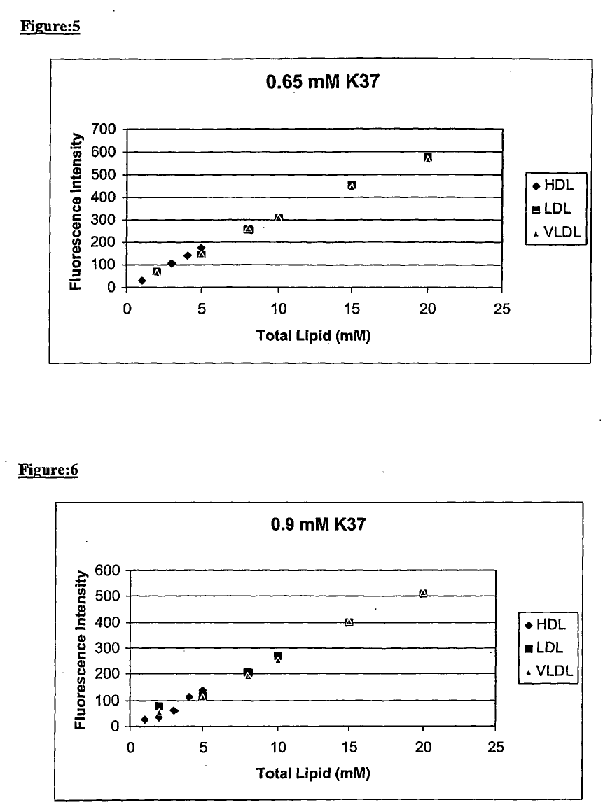

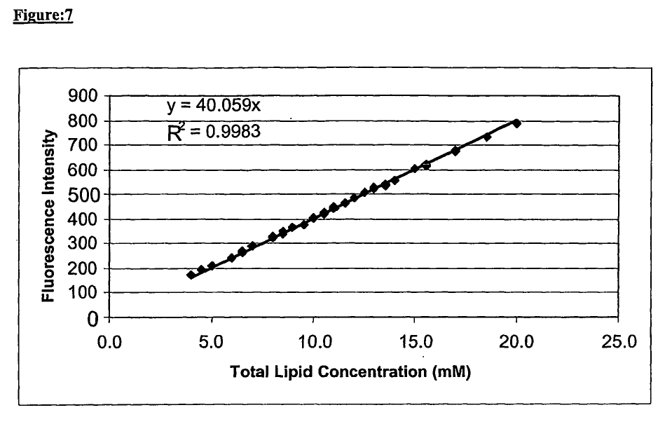

FIG. 6 is a graph showing fluorescence intensity against total lipid concentration for 0.9 mM K-37 in HDL, LDL, and VLDL as referred to in Example 1;

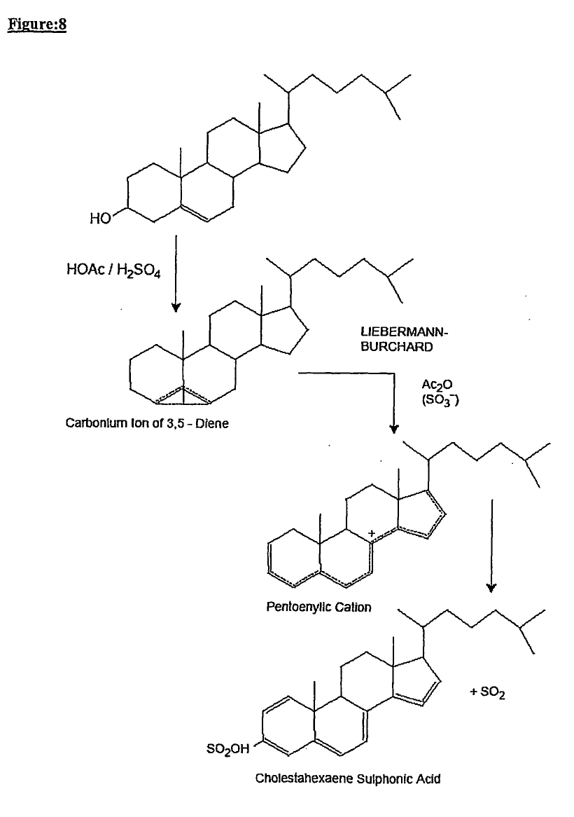

FIG. 7 is a graph showing fluorescence intensity against total lipid concentration for 0.65 mM K-37 in a series of HDL, LDL, and VLDL mixtures as referred to in Example 1;

FIG. 8 shows a schematic for the Liebermann-Burchard reaction as referred to in Example 2;

FIG. 9 shows the absorbance spectrum for the product of the Liebermann-Burchard reaction as referred to in Example 2;

FIG. 10 shows the fluorescence emission spectrum for the product of the Liebermann-Burchard reaction as referred to in Example 2;

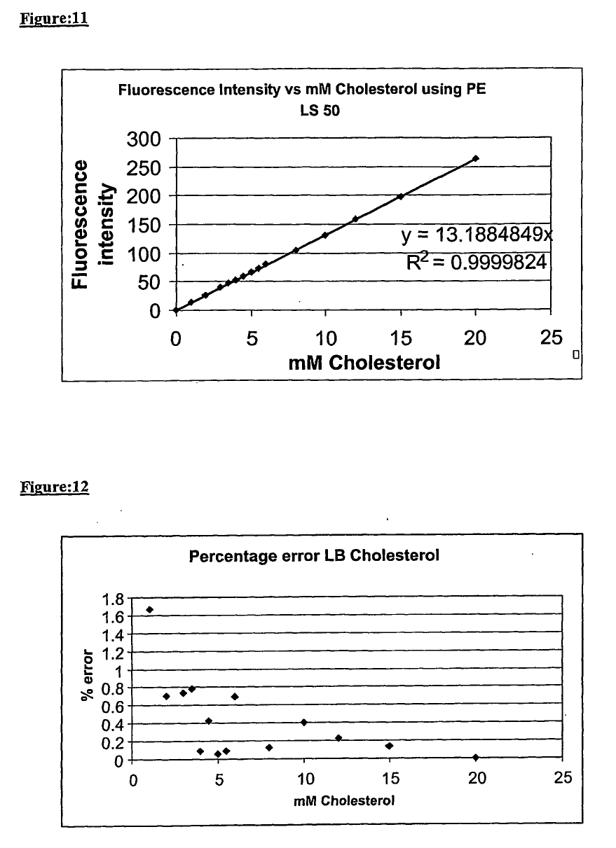

FIG. 11 is a graph showing fluorescence intensity against the concentration of cholesterol as referred to in Example 2;

FIG. 12 is a graph showing percentage error for determining the concentration of cholesterol as referred to in Example 2;

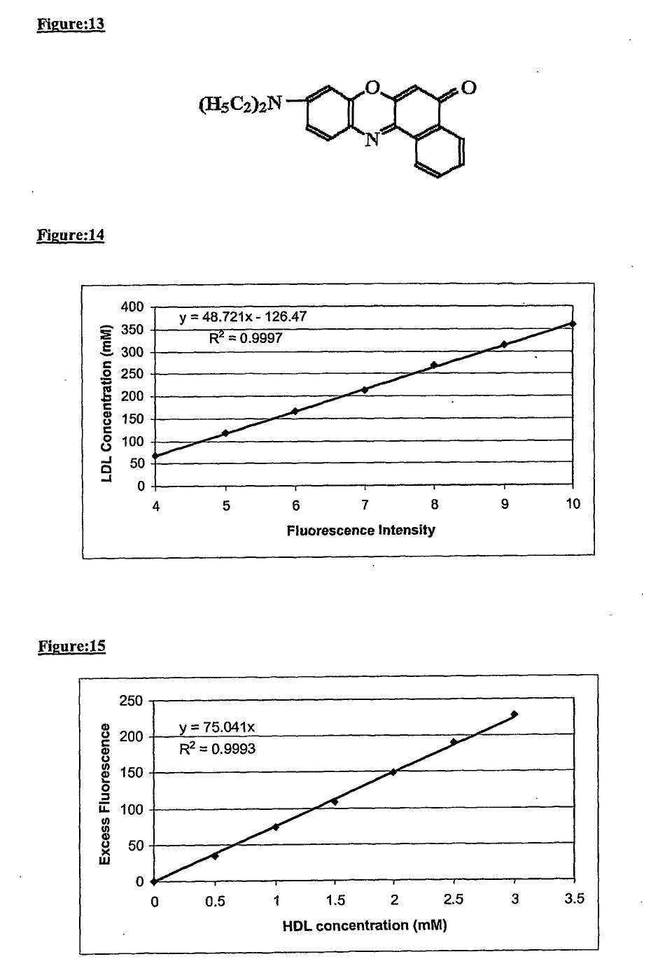

FIG. 13 shows the structure of the dye Nile Red as referred to in Example 4;

FIG. 14 is a calibration curve of LDL concentration against fluorescence intensity as referred to in Example 4;

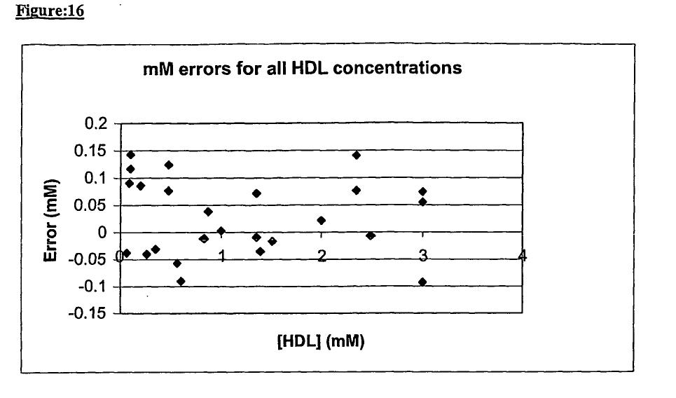

FIG. 15 is a calibration curve of excess fluorescence against HDL concentration as referred to in Example 4 as referred to in Example 4;

FIG. 16 is a graph showing errors against HDL concentration as referred to in Example 4;

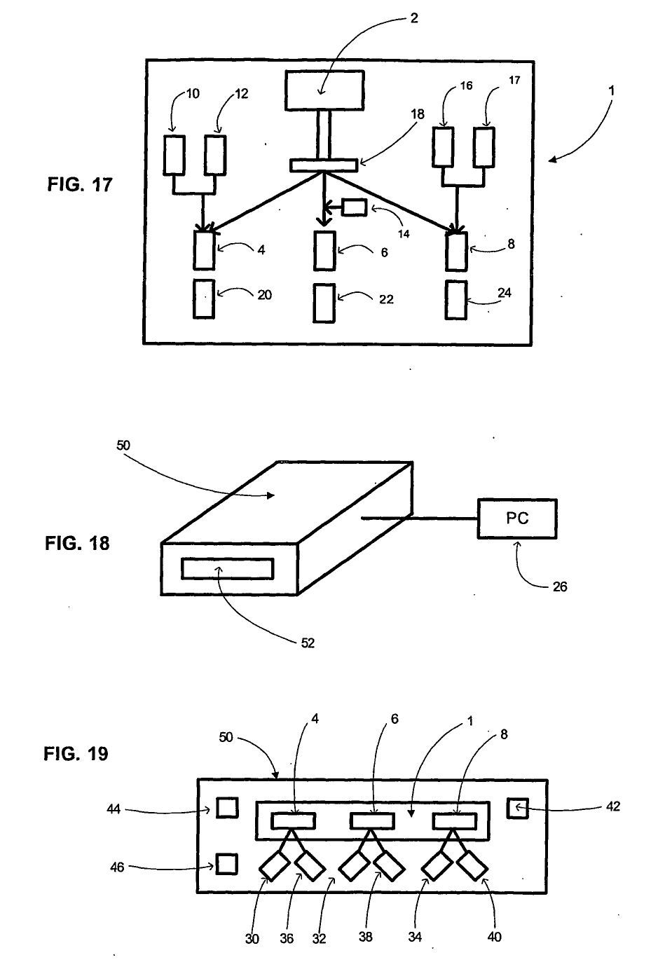

FIG. 17 shows a schematic view of an embodiment of a cartridge according to the invention as referred to in Example 5;

FIG. 18 shows a perspective view of an embodiment of a reader according to the invention as referred to in Example 5;

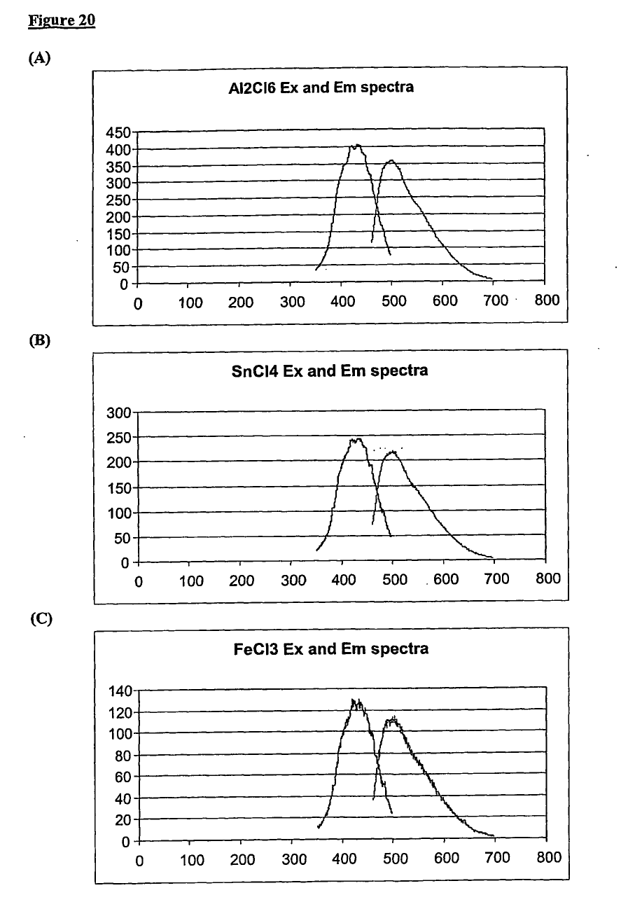

FIG. 19 shows a front view of the cartridge inserted in to the reader as referred to in Example 5;

FIG. 20 illustrates the excitation spectra (Ex—dark trace) for the light source and emission spectra (Em—lighter trace) for products of the L-B Assay when the Lewis acids Al₂Cl₆ (A); SnCl₄ (B) and FeCl₃ (C) were utilised in the method of the invention as referred to in Example 6;

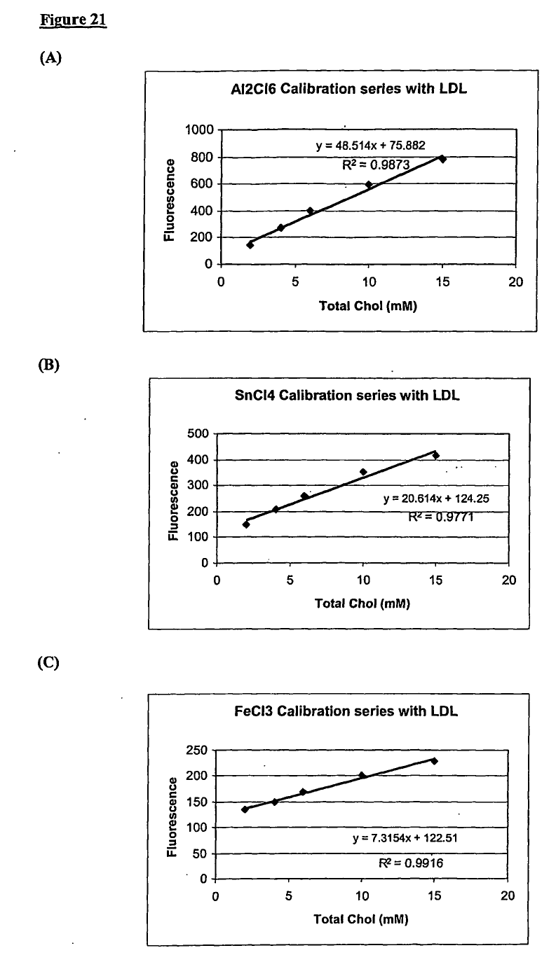

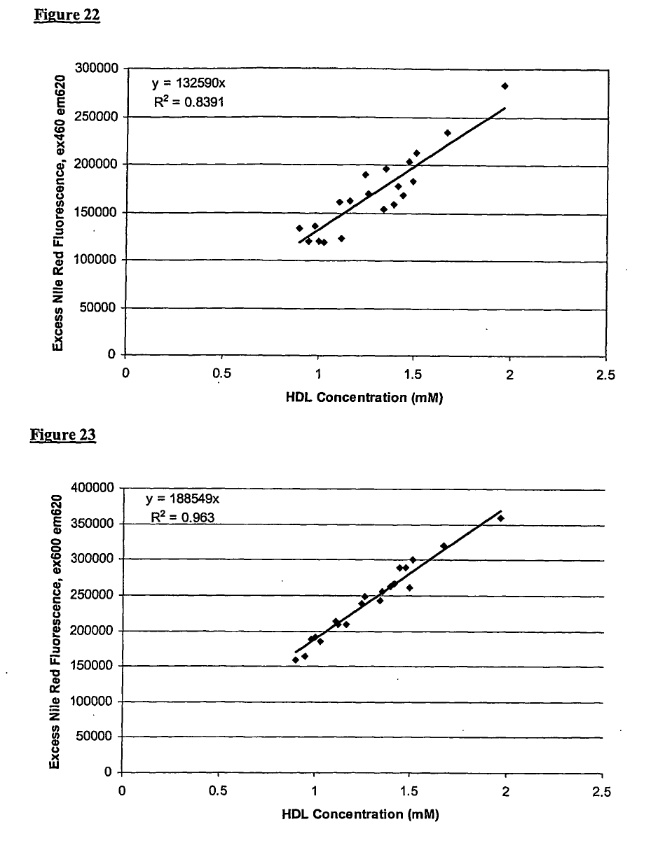

FIG. 21 represents calibration graphs showing fluorescence intensity against the concentration standard cholesterol (in the form of LDL) when utilising Lewis acids, Al₂Cl₆ (A); SnCl₄ (B) and FeCl₃ (C) instead of sulphuric acid in the L-B reaction respectively as referred to in Example 6.

FIG. 22 is a graph illustrating Nile Red fluorescence (ex460 nnm and em620 nm) against HDL concentration as referred to in Example 7;

FIG. 23 is a graph illustrating Nile Red fluorescence (ex600 nnm and em620 nm) against HDL concentration as referred to in Example 7; and

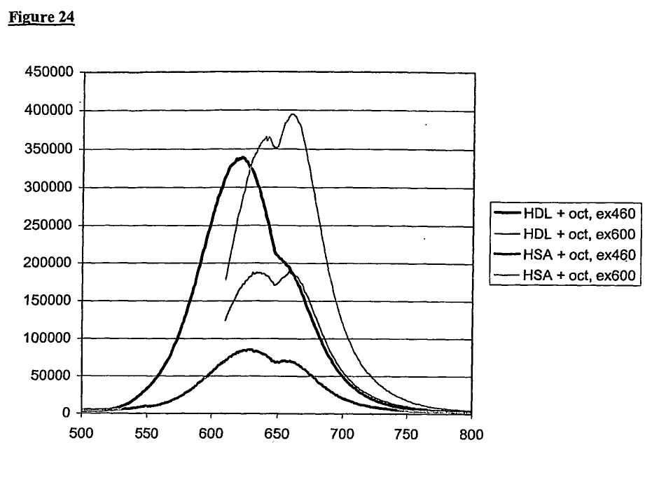

FIG. 24 is a graph illustrating a spectral analysis of Nile Red fluorescence in the presence of HDL (+octanoate) or HSA(+octanoate) at excitation wavelengths of 460 nm and 600 nm as referred to in Example 7.

The inventors carried out a series of experiments in order to investigate the use of fluorescent probes to determine the concentrations of lipoproteins and cholesterol in a blood sample in order to generate a lipid profile for a patient. Knowledge of the lipid profile for a patient, in particular, the levels of LDL, triglycerides. and HDL in the sample, would be advantageous in helping a clinician decide upon a particular course of treatment. The results of these experiments, which are described in the following examples, were then used to develop the method and apparatus according to the invention.

### EXAMPLE 1

### Measurement of Total Lipoprotein Concentration

The inventors investigated the use of the fluorescent dye K-37 to detect the concentration of total lipoproteins (which equates to total triglycerides plus cholesterol plus cholesterol esters as it is assumed that all lipids are bound to lipoproteins) in a series of samples. The dye K-37 is known to the skilled technician, and is readily available. The dye is first excited at a defined wavelength, and then fluorescence is measured at another defined wavelength as described below. The intensity of the fluorescence is used to calculate the concentration of total lipoproteins in the sample (i.e. step (i) of the method according to the invention).

### Method

The dye, K-37, which was dissolved in dimethyl formamide (DM), was added at a range of different concentrations, to a concentration series of HDL, LDL, and VLDL dissolved in phosphate buffered saline. The objective of the experiment was to obtain a linear and equal relationship between fluorescence and lipoprotein concentration for each particle type (HDL, LDL, and VLDL), across the range of lipoprotein concentrations that would be encountered in real plasma or serum samples. Fluorescence intensity was measured in a Perkin-Elmer LS50 fluorimeter, at an excitation wavelength of 450 nm, and at an emission wavelength of 540 nm.

### Results

FIGS. 1 to 3 illustrate the fluorescence intensity versus total lipoprotein concentration for K-37 at three concentrations, i.e. 0.4 mM, 0.65 mM, and 0.9 mM, in HDL, LDL, and VLDL in phosphate buffered saline. The R² values are shown for linear fits to each series (0.4 mM at the top, 0.65 mM in the centre, and 0.9 mM below). The same data are also plotted in FIGS. 4 to 6, and are grouped by K-37 concentration.

The conclusions from the experiment are that:—

1) For all three lipoprotein particle types (HDL, LDL, and VLDL), the R² shows that there is a good linear relationship between total lipoprotein concentration and fluorescence intensity at a K-37 concentration of 0.65 mM. Good linear relationships are also observed for 0.9 mM K-37 in LDL and VLDL, but the linearity at 0.9 mM K-37 in HDL is a little poorer. Linearity is poorer for all lipoproteins with 0.4 mM K-37. It is noteworthy that while it still works at concentrations where linearity is poorer, it is less accurate. However, non-linearities may be dealt with using polynomial fitting.
2) Two factors are thought to affect linearity. At a low dye concentration, there is a flattening off of the response at high total lipoprotein concentration. While the inventors do not wish to be bound by any hypothesis, they believe that this occurs because there is insufficient dye available to fully occupy the lipoprotein particles. At high dye concentrations, there is a flat response with low total lipoprotein concentration. This is caused by self-quenching of the fluorescence when the dyes are packed very closely in the particles.
3) A K-37 concentration of 0.65 mM gives linear and very similar fluorescence responses for all the lipoprotein particle types across the appropriate range when measured in phosphate buffered saline.

Accordingly, 0.65 mM K-37 was then added to a series of HDL/LDL/VLDL mixtures, and fluorescence intensity was measured as described above. The data is illustrated in FIG. 7. As can be seen, the total lipoprotein concentration is highly correlated with fluorescence intensity (R²=0.9983), confirming that this concentration of K-37 (0.65 mM) is suitable for highly accurate measurements of total lipoprotein concentration. When applying this to biological samples from patients, the inventors observed some curvature at high lipid concentration. Consequently a concentration of 0.7 mM K-37 was chosen as the optimal K-37 concentration for use in serum or plasma. Hence, this concentration was selected as the most suitable concentration for the method according to the invention.

### EXAMPLE 2

### Measurement of Total Cholesterol

The inventors then investigated whether it would be possible to use fluorescence measurements to determine the concentration of total cholesterol in a sample (step (ii) of the method according to the invention). This would be in contrast to measuring absorbance as in the conventional assay for cholesterol.

### Method

The method according to the invention was similar to the conventional Liebermann-Burchard reaction assay (L-B), in that the same reagents are used. FIG. 8 illustrates the Liebermann-Burchard reaction. Referring to FIG. 9, there is shown the absorbance spectrum of the L-B product. It can be seen that the absorbance spectrum shows a broad absorbance range, between 400 and 700 nm, which is why absorbance measurements are made with conventional assays to measure cholesterol concentrations in a sample.

However, instead of measuring the absorbance of the coloured product at 550 nm or 600 nm as in the conventional L-B reaction assay and its variations, in the present invention, fluorescence is measured. Referring to FIG. 10, there is shown the fluorescence emission spectrum of the L-B product. Excitation at wavelengths that are responsible for the colour (i.e. 550 nm to 700 nm) do not contribute to this. However, when an excitation wavelength of 450 nm was selected, as this is used in the other assays described herein, the fluorescence surprisingly extends over the range 470-600 nm.

The advantages of measuring fluorescence instead of measuring absorbance are increased sensitivity and decreased volume requirement. The modified procedure presently uses 50 microlitres of plasma and 1 ml of reagent. This is because a conventional 1 cm pathlength cuvette can be used for the fluorescence measurement. However, reagent volumes in the region of ten microlitres would easily be possible. This could not be achieved using absorbance, as the cell pathlength would be too short for accurate measurements (an absorbance of at least 0.01 Au would be required for accuracy). Unless otherwise indicated, fluorescence measurements were carried out in a Perkin-Elmer LS-50 luminescence spectrometer.

### Measurement of Total Cholesterol

Calibration standards with total cholesterol range between 0 and 20 mM were made up from characterised LDL samples supplied from Prof. Chris Packard's lab (Glasgow). 50 microlitres of a sample were added to 1 ml of L-B reagent, and incubated for 5 minutes at room temperature (although a shorter or longer incubation time may be sufficient for a successful measurement). Fluorescence of each sample was measured using an excitation wavelength of 450 nm, and an emission wavelength of 540 nm. Fluorescence was plotted against total cholesterol concentration as illustrated in FIG. 11. The closeness of the correlation coefficient R² to 1 shows the high degree of linearity of the measurement.

The gradient of the fitted line was used to calculate total cholesterol from the fluorescence of each sample. Percentage errors between actual and measured total cholesterol are shown in FIG. 12. The results show that the fluorescence L-B assay can be used for measurement of serum cholesterol with very high accuracy in the range from zero to 20 mM, which covers the range that would be expected from clinical samples.

Therefore, the inventors realised that it would be possible to excite a blood sample at 450 nm, and measure the emission at 540 nm, in order to simultaneously determine the concentration of both total lipoprotein (step (i) of the method) and cholesterol (step (ii)) in the sample. As a result of the experiments conducted in Examples 1 and 2, the inventors had developed the method according to the first aspect for generating a lipid profile consisting of the concentrations of total lipoprotein and cholesterol in the sample. This is a marked improvement over currently available assays, which require the use of two completely independent assays.

### EXAMPLE 3

### Blocking of HSA

The inventors then conducted further investigations to optimise the method according to the invention. To this end, they realised that Human Serum Albumin (HSA), which is a major component of blood plasma, has hydrophobic binding sites to which K-37 binds and fluoresces. This additional fluorescence of K-37 when bound to HSA causes a substantial background signal, which distorts and thereby causes significant errors in the measurement of the lipoprotein molecules, i.e. HDL, LDL and VLDL. They therefore decided to block the hydrophobic binding sites on HSA with a ligand binding inhibitor, such as sodium octanoate, to see if the additional fluorescence could be minimised. It was envisaged that inhibiting the binding of K37 with HSA in this way would improve the accuracy of the results obtained using K37 fluorescence measurements.

### Methods

The dye K37 was added at a concentration of 0.5 mM to LDL at a total lipid concentration of 5 mM, in the presence and absence of 50 mg/ml HSA. Measurements were made with and without the addition of 0.1 M sodium octanoate, which acted as a ligand binding inhibitor.

### Results

Fluorescence intensity was measured for all samples and is summarised in the Table below:

| Sample | Fluorescence Intensity |
|---|---|
| K-37 plus 5 mM LDL | 213500 |
| K-37 plus 50 mg/ml HSA | 79300 |
| K-37 plus 5 mM LDL + octanoate | 209700 |
| K-37 plus 50 mg/ml HSA + octanoate | 3600 |

The results show that the fluorescence intensity of K-37 in LDL alone is 213500 units. The fluorescence intensity of K-37 when octanoate is added to LDL is 209700 units (i.e. about the same as without octanoate), which suggests that the presence of octanoate does not contribute to the fluorescence intensity of K-37 bound to LDL by itself. The fluorescence intensity of K-37 bound to HSA is 79300 units, whereas that of K-37 in the present of HSA and octanoate is 3600. This illustrates that HSA contributes to K-37 fluorescence and is therefore an interfering signal. The addition of octanoate significantly reduces the interference and thereby obviates the disruptive effects of HSA in the sample. The results therefore show a large suppression of fluorescence intensity for K-37 with HSA in the presence of octanoate, but little effect on K37 fluorescence in LDL. This showed that octanoate is remarkably successful at blocking the K-37 binding site on HSA, making the K-37 fluorescence a true measure of total lipoprotein concentration.

Accordingly, the inventors believe that a ligand binding inhibitor such as octanoate, which can bind to the hydrophobic binding sites of HSA, can be added to the blood sample prior to measuring the fluorescence of K-37 to improve the accuracy of the total lipoprotein concentration. In addition, the inventors suggest that this technique can also be used to block the binding of other ligands (e.g. Nile Red probe) to the hydrophobic binding sites of HSA, and to displace ligands that may be already bound thereto, and which have a lower affinity for HSA than the octanoate. Subsequent to this work the inventors found that 0.7 mM K37 and 100 mM octanoate were optimal.

### EXAMPLE 4

### Measurement of HDL

The inventors then investigated whether it would be possible to distinguish between the different types of lipoprotein present in a blood sample. Hence, they tested the efficacy of using fluorescent probes, other than K-37, for example, Nile Red, to see if the lipoprotein types were distinguishable. To their surprise, they found that by using Nile Red instead of K-37, it was possible to determine the concentration of HDL in a blood sample.

### Method

The principle of the measurement is that the probe Nile Red is more fluorescent in HDL than in LDL, and VLDL. The structure of Nile Red is illustrated in FIG. 13. The measurement is more complicated than the K37 measurement for total lipoprotein, as a calculation must be made of excess fluorescence from Nile Red in HDL, and not simply total fluorescence of all lipoproteins. The procedure is as follows:—

### 1) Calibration

0.5 mM Nile Red dissolved in dimethylformamide was mixed with LDL at varying total lipoprotein concentrations usually between 4 and 10 mM (typically 50 microlitres of dye are mixed with 50 microlitres of lipoprotein and 1 ml of phosphate buffered saline). Samples were put in a fluorimeter and fluorescence intensity was measured (excitation wavelength 450 nm, emission wavelength 600 nm). Fluorescence intensity was plotted against LDL total lipid concentration, giving a straight calibration line with slope “X” and intercept “Y”, as shown in FIG. 14.

The procedure was then repeated for mixtures of LDL and HDL. HDL was added at concentrations of between 0 and 3.0 mM, with LDL added to keep the total lipoprotein concentration at 5 mM for all samples. Fluorescence intensities for these samples were then measured. A plot was then made of excess fluorescence due to the presence of HDL, giving a straight calibration line having slope “Z”, as illustrated in FIG. 15.

### 2) Measurements of Unknowns

0.5 mM Nile Red dissolved in dimethylformamide was mixed with the sample under investigation after pre-treatment with octanoate. The sample was put into a fluorimeter and fluorescence intensity was measured under the same conditions as for the calibration described above.

### 3) Calculation of HDL Concentration

Calculation of HDL requires knowledge of the total lipoprotein concentration “A”, which can be measured from the fluorescence intensity of K37. For a particular sample, the fluorescence intensity that would be expected if the sample contained no HDL is obtained from the calibration line shown in FIG. 14. The measured fluorescence intensity minus this calculated fluorescence intensity is the excess fluorescence due to HDL present in the sample.

The HDL concentration “C” in the unknown sample can then be obtained using the calibration line shown in FIG. 15 and the following equation:—

C=(B−(AX−Y))/Z

A range of concentrations of HDL/LDL/VLDL mixtures were prepared intended to cover the range of concentrations that would be expected in real clinical samples. The calibration data discussed above were used to calculate HDL concentrations from the mixtures. FIG. 16 illustrates errors between actual HDL concentration and HDL concentration determined from Nile Red fluorescence, showing a maximum error of only approximately 0.15 mM.

As a result of these data, the inventors have shown that it is possible to distinguish between the types of lipoprotein present in a sample, and to determine the concentration of HDL using the dye Nile Red.

Following the findings described in Example 3, concerning the addition of octanoate to block the hydrophobic binding sites of HSA, the inventors then observed that Nile Red also binds to HSA (the same hydrophobic binding sites as K37) and fluoresces. This additional fluorescence of Nile Red when bound to HSA also causes a substantial background signal, which distorts and thereby causes significant errors in the measurement of HDL. They therefore decided to block the hydrophobic binding sites in HSA with the same ligand binding inhibitor as for K-37 blocking, i.e. sodium octanoate. The experiments conducted with Nile Red and HSA, were based on those discussed in Example 3, and all using 0.5 mM Nile Red.

| Sample | Fluorescence Intensity |
|---|---|
| Nile Red plus 5 mM LDL | 187.532 |
| Nile Red plus 50 mg/ml HSA | 58.905 |
| Nile Red plus 5 mM LDL + 50 mM octanoate | 183.786 |
| Nile Red plus 50 mg/ml HSA + 50 mM octanoate | 9.118 |
| Nile Red plus PBS + 50 mM Octanoate | 7.382 |

The results show that the fluorescence intensity of Nile Red in LDL alone is 187.532 units. The fluorescence intensity of Nile Red when octanoate is added to LDL is 183.786 units (i.e. about the same as without octanoate), which suggests that the presence of octanoate does not contribute to the fluorescence intensity of K-37 bound to LDL by itself. The fluorescence intensity of Nile Red bound to HSA is 58.905 units, whereas that of Nile Red in the present of HSA and octanoate is 9.118. This illustrates that HSA contributes to Nile Red fluorescence and is therefore an interfering signal. The addition of octanoate significantly reduces the interference and thereby obviates the disruptive effects of HSA in the sample. The results therefore show a large suppression of fluorescence intensity for Nile Red with HSA in the presence of octanoate, but little effect on Nile Red fluorescence in LDL.

This showed that octanoate is remarkably successful at blocking the Nile Red binding site on HSA, making the Nile Red fluorescence a true measure of lipoprotein concentration. Accordingly, the inventors believe that a ligand binding inhibitor such as octanoate, which can bind to the hydrophobic binding sites of HSA, can be added to the blood sample prior to measuring the fluorescence of Nile Red to improve the accuracy of the lipoprotein (HDL) concentration. Subsequent to this work the inventors found that 0.4 mM Nile Red and 50 mM, or more preferably about 100 mM, octanoate were optimal for the analysis of serum samples.

### EXAMPLE 5

### Simultaneous Assay to Generate Lipid Profile

Examples 1 and 3 illustrate how fluorescence measurements of the dye K-37 may be used to determine the concentration of total lipoproteins in a sample according to step (i) of the method of the invention.

Example 2 describes how fluorescent measurements (i.e. not absorbance) of the product of the L-B assay may be used to determine the concentration of total cholesterol in the sample according to step (ii) of the method of the invention. Fortuitously, as with K37, the product of the L-B assay may be excited at a wavelength of about 450 nm and its emission may also be measured at about, or above, 540 nm.

Examples 4 illustrates, in view of total lipoprotein measurements from step (i), how the fluorescence measurements of Nile Red may be used to determine the concentration of HDL in a sample according to step (iii) of the method of the invention.

Therefore, the inventors realised that it is possible to create a single fluorescence-based method for analysing the lipid composition of a patient's blood sample in order to create a lipid profile for that patient. The preferred method consists of three assays, all of which can be carried out under very similar conditions, and hence, can produce results very quickly. The clinician may use this information to decide upon a course of treatment.

The following describes how the inventors developed the method according to the first aspect of the invention such that a lipid profile may be quickly generated from a single sample which is subjected to three simultaneous fluorescence based assays.

### Method

A blood sample is initially taken from a patient, and then centrifuged using well-established conventional techniques, in order to separate the serum. The serum is then separated in to three aliquots (a, b & c), each of which is subjected to biochemical analysis to determine the concentration of a lipid component. Aliquot (a) is used to determine the concentration of total lipoprotein; aliquot (b) is used to determine the concentration of cholesterol; and aliquot (c) is used to determine the concentration of HDL, as described below.

Aliquot (a)—The HSA ligand binding inhibitor, sodium octanoate, is added to the 2 ml of PBS to a concentration of 50 mM as described in Example 3 above. 25 microlitres of serum aliquot (a) are then added to the PBS/Octanoate solution. The probe K-37, which was dissolved in dimethyl formamide (DMF), was then slowly added under stirring to the sample to a final concentration of 0.65 mM. The sample was then excited at 450 nm in order to cause the probe to fluoresce. The fluorescence was measured at an emission wavelength of 540 nm, and from this value it was then possible to determine the concentration of total lipoprotein (HDL, LDL, and VLDL) in the sample, as described in Example 1 above.

Aliquot (b)—10 microlitre of serum was added to 2 ml of L-B reaction reagents (60% acetic anhydride, 30% acetic acid and 10% sulphuric acid). The sample was then excited at 450 nm in order to cause the product of the L-B reaction to fluoresce. The fluorescence was measured at an emission wavelength of 540 nm, and from this value it was then possible to determine the concentration of cholesterol in the sample as described in Example 2 above.

Aliquot (c)—The HSA ligand binding inhibitor, sodium octanoate, is added to 2 ml of PBS to a concentration of 50 mM as described in Example 4 above. 25 microlitres of serum aliquot (c) are then added to the PBS/Octanoate solution. The probe Nile Red was then slowly added under stirring to the sample to a final concentration of 0.5 mM. The sample was then excited at 450 nm in order to cause the probe to fluoresce. The fluorescence was measured at an emission wavelength of 600 nm, and from this value it was then possible to determine the concentration of HDL in the sample as described in Example 4 above.

It will be appreciated that the fluorescence measurements for all three aliquots are carried out under similar conditions. Therefore, an advantage of the method according to the invention is that all three aliquots may be analysed simultaneously by the same instrument. Accordingly, use of the above method quickly generates a lipid profile consisting of (a) the total lipoprotein concentration, (b) the total cholesterol concentration, and (c) the concentration of HDL in the sample.

The triglyceride concentration may be easily calculated by subtracting the values of the cholesterol concentration determined by assaying aliquot (b) from the total lipoprotein concentration determined by assaying aliquot (a). Therefore, the lipid profile generated now also includes the concentration of triglycerides, which assists the clinician treating the patient.

With a knowledge of total cholesterol concentration determined by assaying aliquot (b), the HDL concentration determined by assaying aliquot (c), and the triglyceride concentration calculated as described above, it is then possible to calculate the concentration of LDL in the sample by inserting the values in to the Friedewald Equation:—

(CH-LDL)=CH−(CH-HDL)−TG/5

where CH is the total cholesterol concentration, (CH-LDL) is the cholesterol LDL concentration, (CH-HDL) is the cholesterol HDL concentration, and TG is the triglyceride concentration. As a result, the lipid profile generated now also includes the concentration of LDL in the sample.

### EXAMPLE 6

### A Device for Generating a Lipid Profile

Having illustrated that lipid profiles many be generated based on the use of three similar fluorescence assays, the inventors proceeded to design an apparatus that may be used to generate lipid profiles according to the second aspect of the invention.

Referring to FIGS. 17-19, there is shown a portable device developed by the inventors, which can be used to generate a patient's lipid profile. The device consists of a cartridge 1, which is shown in detail in FIG. 17, and a reader 50, which is shown in FIG. 18.

The cartridge 1 has a series of interconnected reservoirs, along which fluids may flow in order to carry out the assays according to the invention. The cartridge 1 plugs into the reader 50 via slot 52 for detecting and measuring fluorescence intensity for each of these assays carried out in the cartridge 1.

Referring to FIG. 17, the cartridge 1 has a sample reservoir 2 in which a biological fluid taken from a patient, such as blood, is contained. A filter 18 is provided for removing blood cells from the blood, leaving plasma or serum or other body fluid, with which the assays are carried out. The fluid is divided in to three aliquots (first, second and third aliquots according to the method of the invention), and urged along channels in to reaction reservoirs 4, 6, 8, respectively.

Two reagent reservoirs 10, 12 containing K-37 and sodium octanoate, respectively, are connected to reaction reservoir 4 (the K-37 reaction reservoir). The dye and octanoate are urged in to reservoir 4 and the total lipoprotein assay is initiated.

Reagent reservoir 14 contains the reagents for the L-B reaction, and so when these are urged in to reaction reservoir 6, they are mixed with the biological fluid, and the cholesterol assay is initiated.

Two reagent reservoirs 16, 17 containing Nile Red; and HSA blocking agents (e.g. sodium octanoate and benzoic acid—see example 8), respectively, are connected to reaction reservoir 8 (for determining HDL). The Nile Red and HSA blocking agents are urged in to reservoir 8, mixed with the fluid, and the HDL assay is initiated.

The cartridge also includes fluorescence standards 20, 22, 24, which may be used to calibrate the reader 50 for steps (i), (ii) and (iii) respectively.

The cartridge 1 plugs into the slot 52 in the front of the reader 50, as shown in FIG. 18. Slotting the cartridge 1 in to the reader 50 causes the three reaction reservoirs 4, 6, 8 to each align with a corresponding light source 30, 32, 34, and a corresponding detection photodiode 36, 38, 40, which are present in the reader. Alternatively, the reader 50 may have only one light source or LED instead of separate LEDs for each reservoir 4, 6, 8.

The LEDs (or guides from an LED) 30, 32, 34 each provide the corresponding assay with the required fluorescence excitation illumination for each assay to fluoresce. The wavelength of the light from each of the LEDs 30, 32 is at about 450-470 nm whereas the wavelength of the light from LED (or guide from an LED) 34 is at about 600 nm. When a white LED is use, the light may pass through a 450 nm or 600 nm interference filter (not shown) to produce the correct excitation wavelengths before it is directed to the appropriate reaction reservoirs. The reader 50 may have an excitation correction system 46. Hence, fluorescence of the three simultaneous assays is collected with lenses or similar collection optics, and may pass through a polariser at a wavelength of 540 nm (for total lipoprotein and cholesterol assays) and at a wavelength of about 620 nm for the third assay (HDL). Alternatively a common polariser may be used for both assays. For measurement of the fluorescence intensity, the current output of the photodiodes 36, 38, 40 is amplified and read as a current or a voltage.

Accordingly, the reader 50 is configured to hold the cartridge 1 to detect and measure the fluorescence intensities of each of the three assays, to thereby generate the lipid profile consisting of total lipoprotein concentration, HDL concentration, and cholesterol concentration. In one embodiment, the apparatus has an LCD readout display 42 on which the concentrations of each of the blood components are shown. In another embodiment the reader 50 may be powered by, and have its readout fed through, a USB port of a PC, laptop, PDA or mobile phone 26 enabling the clinician to readout information on the concentration of triglycerides, HDL and also IDL as described in Example 5. Alternatively, the apparatus may embody both aspects of the cartridge and measurement instrument and comprise a microprocessor 44 which can calculate the concentrations of each of the lipid components itself, including the Friedewald equation automatically.

Advantages of the cartridge 1 and reader device 50 reside in the quick and easy assay systems, which can be carried out simultaneously to generate the lipid profile from the biological fluid. The clinician can then have knowledge of the clinically important lipids (e.g. LDL concentration) and then decide on an effective course of treatment.

The cartridge 1 is disposable and may be cheaply made. The cartridge 1 can be formed with the reagents sealed within the appropriate reagent reservoirs 10, 12, 14, 16, 17 thereby avoiding the inconvenience of reagent preparation and even contact with reagents.

### EXAMPLE 6

### Alternative Strong Acids for Use in Step (ii)

The inventors repeated the experiments described in Example 2 to illustrate that strong acids, other than sulphuric acid, may be used to measure cholesterol concentrations in the second aliquot of the a sample according to the method of the invention. The inventors therefore conducted experiments utilising Lewis acids.

The inventors first studied the spectra produced from products of the L-B assay in which the Lewis acids, Al₂Cl₆, SnCl₄ and FeCl₃ were used in the reaction instead of sulphuric acid. The final concentration of each acid was 1.8M (as for the sulphuric acid in Examples 2). FIG. 20 illustrates the excitation spectra (Ex—dark trace) for the light source and emission spectra (Em—lighter trace) for products of the L-B Assay when the Lewis acids Al₂Cl₆ (A); SnCl₄ (B) and FeCl₃ (C) were used. The emission spectra were equivalent to that obtained when utilising sulphuric acid. This illustrates that other strong acids, in this case Lewis acids, may be utilised in the L-B assay when conducting assays according to the invention.

FIG. 21 represents calibration graphs showing fluorescence intensity against standard cholesterol concentrations (in the form of LDL) when utilising Lewis acids, Al₂Cl₆ (A); SnCl₄ (B) and FeCl₃ (C) instead of sulphuric acid in the L-B reaction. The linearity of these graphs illustrate that reliable measurements of cholesterol concentration can be obtained when a Lewis acid is use in the L-B reaction according to the method of the invention.

### EXAMPLE 7

### Further Optimisation of the Step (iii) According to the Method of the Invention

Further tests were performed on human serum samples to investigate optimum excitation wavelengths for inducing fluorescence indicative of HDL levels according to the method of the invention.

The inventors tested a number of wavelengths and have established that, when using Nile Red, that an excitation wavelength of 600 nm and an emission wavelength of 620 nm gives optimal results (see FIG. 22). The inventors were surprised that this excitation wavelength was optimal because it is to the very long wavelength edge of the spectrum.

For certain samples the inventors observed a noisier plot with an excitation wavelength of 460 nm and an emission wavelength of 620 nm (see FIG. 23).

The inventors believe that Nile Red is about 5 times more fluorescent in HDL than VLDL and LDL when excited at 600 nm as opposed to excitation at 460 nm where it is only about 2 times more fluorescent. This gives a better signal to noise when subtracting from the standard curve of LDL plus VLDL.

Although the inventors do not wish to be bound by any hypothesis, they believe the “noise” observed in serum samples, excited at 460 nm, is an effect of signal-to-noise. The inventors have noted that Nile Red binds to HSA and particularly at low lipid concentrations. They therefore performed a spectral analysis of Nile Red fluorescence in the presence of HDL (+octanoate) or HSA (+octanoate) both at an excitation wavelength of 460 nm and 600 nm (see FIG. 24). These experiments resulted in unexpected spectral behaviour which the inventors believe may be explained by the fact that Nile red is in a rigid but polar environment (binding site on HSA) and the Nile red exhibits twisted intramolecular charge transfer (TICT) (Journal of Photochem and Photobiol A:Chemistry 93 (1996) 57-64) that shifts the excitation and emission to longer wavelengths. The molecule in this excited state has a different dipole moment and so behaves like a different species. In exciting at 600 nm the better signal-to-noise due to the larger difference in signal between Nile Red in HDL compared with other lipoproteins more than compensates for the excitation of the TICT state because TICT fluorescence is excluded by the 620 nm emission wavelength setting. In other words, while we excite the HSA/Nile Red more optimally at 600 nm its fluorescence is rejected by the instrument.

This lead the inventors to realise that the HDL/Nile red assay may be improved further by using an additional blocker. They tried agents that block the drug binding domain of HSA. To their surprise they found that agents such as benzoic acid, and its trichoro and triiodo derivatives, all worked to displace the Nile Red from HSA without affecting the lipoprotein fluorescence at about 5 mM. The benzoic acid has the added bonus of quenching the Nile Red residual fluorescence in solution by about 20%.

### EXAMPLE 5

### Simultaneous Assay to Generate Lipid Profile

Examples 1 and 4 illustrate how fluorescence measurements of the dye K-37 may be used to determine the concentration of total lipoproteins in a sample according to step (i) of the method of the invention.

In addition, Examples 2 and 6 describe how fluorescent measurements (i.e. not absorbance) of the product of the L-B assay may be used to determine the concentration of total cholesterol in the sample according to step (ii) of the method of the invention. Fortuitously, as with K37, the product of the L-B assay may be excited at a wavelength of about 450 nm and it's emission may also be measured at about, or above, 540 nm.

Furthermore Examples 4 and 7 illustrate, in view of total lipoprotein measurements from step (i), how the fluorescence measurements of Nile Red may be used to determine the concentration of HDL in a sample according to step (iii) of the method of the invention.

Therefore, the inventors realised that it is possible to create a single fluorescence-based method for analysing the lipid composition of a patient's blood sample in order to create a lipid profile for that patient. The preferred method consists of three assays, all of which can be carried out under very similar conditions, and hence, can produce results very quickly. The clinician may use this information to decide upon a course of treatment.

1. A method of generating a lipid profile for a sample solution, the method comprising the steps of:—
(i) determining the concentration of total lipoprotein in a first aliquot of the sample using fluorescence analysis;
(ii) determining the concentration of total cholesterol in a second aliquot of the sample using fluorescence analysis; and
using the concentrations of the total lipoprotein, and of total cholesterol to generate a lipid profile.

2. The method according to claim 1 wherein the sample is a biological fluid.

3. The method according to claim 2 wherein the biological fluid is blood serum or plasma, or lymph.

4. The method according to any preceding claim wherein step (i) comprises adding to the sample a probe substance which binds to lipoproteins, and which when bound thereto, fluoresces under appropriate excitation.

5. The method according to claim 4 wherein the probe substance is 4-dimethylamino-4′-difluoromethyl-sulphonyl-benzylidene-acetophenone (DMSBA or K-37).

6. The method according to any preceding claim wherein step (ii) comprises adding Liebermann-Burchard assay (L-B) reagents to the second aliquot and measuring fluorescence after appropriate excitation.

7. The method according to any one of claims 4-6 wherein a ligand binding inhibitor that substantially inhibits the binding of the probe substance to Human Serum Albumin is added to the first aliquot of the sample before lipoprotein concentrations are determined.

8. The use according to claim 7 wherein the ligand binding inhibitor is octanoate.

9. The method according to any preceding claim further comprising the further step of:
(iii) determining the concentration of a particular class, or sub-class of lipoprotein in a third aliquot of the sample using fluorescence analysis; and
using the concentrations of the total lipoprotein; total cholesterol and of the particular class or sub-class of lipoprotein to generate a lipid profile.

10. The method according to claim 9 wherein the step (iii) comprises adding to the third aliquot of the sample a second probe substance which binds to HDL, and which when bound thereto, fluoresces under appropriate excitation.

11. The method according to claim 10 wherein the second probe substance is Nile Red.

12. The method according to any one of claims 9-11 wherein a ligand binding inhibitor according to claims 7 or 8 is added to the third aliquot.

13. The method according to any one of claims 9-12 wherein an agent that blocks the drug binding domain of HSA is also added to the third aliquot before HDL concentrations are determined.

14. The method according to claim 13 wherein the agent is benzoic acid or a salt or derivative thereof.

15. The method according to any preceding claim wherein, for each step, the fluorescence is induced by exciting the sample at an excitation wavelength of between about 400 nm-500 nm, and the resultant fluorescence measured at an emission wavelength of between about 500-650 nm.

16. An apparatus for generating a lipid profile for a sample solution, the apparatus comprising at least one reaction reservoir for conducting cholesterol and lipoprotein assays; containment means adapted to contain reagents required for the method according to any one of claims 1-15; excitation means operable to excite the sample so that it fluoresces, and detection means operable to detect the fluorescence emitted by the sample.

17. The apparatus according to claim 16 wherein the apparatus comprises a first reaction reservoir in which an assay to determine the concentration of total lipoprotein in the sample may be conducted; a second reaction reservoir in which an assay to determine the concentration of total cholesterol in the sample may be conducted; and a third reaction reservoir in which an assay to determine the concentration of HDL in the sample may be conducted.

18. The apparatus according to either claim 16 or 17, wherein the apparatus comprises a reader and a cartridge, which cartridge comprises the at least one reaction reservoir, and the containment means.

19. The apparatus according to any one of claims 16-18 further comprising processing means adapted to determine the concentration of total lipoprotein and total cholesterol in the sample based on the fluorescence detected and optionally for determining the concentration of HDL in the sample.

20. The apparatus according to claim 19 wherein the processing means may be operable to: (a) calculate the concentration of triglyceride in the sample by subtracting the concentration of cholesterol from total lipoprotein; and/or (b) calculate the concentration of LDL in the sample using the Friedewald equation.

21. The apparatus according to any one of claims 16-20 further comprising display means for displaying a lipid profile for the sample.
